SEQUENCE LISTING (28 sequences)

SEQ ID NO: 1 - protein, 831 aa, Carboxydothermus pertinax
MGKVVLVDGNSLLHRAFFALPPLKTSKGEPTGAVYGFLTMLFKVIKDENPDYLAVAFDVSRKTFRSDQFSAYKGHRKEAPEELVPQFALVREVLKVLNIPYIELMGYEADDIIGHLSKVFVNHGHEVKIYTADRDMLQLVEEKVAVYLTKKGITDLVKMDLAKVQENYGLKPIQLIDVKGLMGDPSDNIPGVPGIGEKTALDLIKTYGSLEEVLTHKEELKPKLKEKLTEHENLAKISKELATILREIPLEISLEDLKVKEPDYEEAAKLFTRLEFKSFLKEVEPKVKKEYQESKETVNIEIIKAEGQVVVVFNDGFYVDDGEKTSFYSLDQLVDLQEIFRGKEIITDDAKGIYRFCLEKGISFPKVNFDARIAAYVLNPADQNPGLNGLYIKYNLPVYDDLFLNIRGLFYLKKEMLAKIREQQQEKLYQEIELPLTPVLARMEFTGIQVDREALKEMSLELGEQMEALTREIYSLAGEEFNLNSPKQLGVILFEKLGLPVIKKTKTGYSTDAEVLEELFAYHEIVGKILNYRQLMKLKSTYTDGLMPLINEYTGKLHTTFNQTGTLTGRLASSEPNLQNIPVRLELGRKLRKMFIPSTGYDYIISADYSQIELRLLAHFSEEPKLIEAYQKGEDIHRKTAAEVFGVPLEEVSLEMRSHAKSVNFGIVYGISDFGLGRDLKIPREVAGKYIKNYFANYPKVREYLDGLIRTAREKGYVTTLFGRRRYIPELTAKNRTVQSFGERTAMNTPLQGTAADIIKLAMINMERELLRKGLKSRLLLSVHDELVLEVPAEEVEEVKSLVKKVMESVVELKVPLIAEVGMGKNWYEAK

SEQ ID NO: 2 - protein, 831 aa, Carboxydothermus ferrireducens
MGKVVLVDGNSLLHRAFFALPPLKTTKGEPTGAVYGFLTMLFRVIKDEKPEYLAVAFDVSRKTFRTKQFTAYKGHRKEAPDELVPQFALVREVLKVLNVPYIELDGYEADDIIGHLSRVFAGQGHEVVIYTADRDMLQLVDEKTVVYLTKKGITELVKMDLAAILESYGLKPKQLVDVKGLMGDPSDNIPGVPGIGEKTALDLIKTYGSVEEVLAHKDELKPKLREKLAEHENLAKISKQLATILREIPLEISLEDLKVKEPNYEEVAKLFLRLEFKSFLKEIEPKIKKEYQEGKDLVQFETVETEGQIAVVFSDGFYVDDGEKTKFYSLDRLNEIQEIFRNKKIITDDAKGIYHVCLEKGLTFPEVCFDARIAAYVLNPADQNPGLKGLYLKYDLPVYEDVSLNIRGLFYLKKEMMKKIFEQEQERLFYEIELPLTPVLAQMEHTGIQVDREALKEMSLELGEQIEELIREIYALAGEEFNLNSPKQLGVILFEKLGLPVIKKTKTGYSTDAEVLEELLPYHEIIGKILNYRQLMKLKSTYTDGLMPLINERTGKLHTTFNQTGTLTGRLASSEPNLQNIPIRLELGRKLRKMFIPSPGYDYIVSADYSQIELRLLAHFSEEPKLIEAYQKGEDIHRKTASEVFGVSLEEVTPEMRAHAKSVNFGIVYGISDFGLGRDLKIPREVAGKYIKNYFANYPKVREYLDELVRTAREKGYVTTLFGRRRYIPELSSKNRTVQGFGERTAMNTPLQGSAADIIKLAMINVEKELKARKLKSRLLLSVHDELVLEVPAEELEEVKALVKGVMESVVELKVPLIAEVGAGKNWYEAK

SEQ ID NO: 3 - protein, 831 aa, Carboxydothermus islandicus
MGKVVLVDGNSLLHRAFFALPPLKTTKGEPTGAVYGFLTMLFRVIRDEKPEYLAVAFDVSRKTFRTEQFTAYKGHRKEAPDELVPQFALVREVLKVLNVPYIELDGYEADDIIGHLSRVFAGQGHEVVIYTADRDMLQLVDEKTVVYLTKKGITELVKMDLAEVLENYGLKPQQLVDVKGLMGDPSDNIPGVPGIGEKTALDLIKTYGSVEEVLARKEELKPKLREKLAEHESLAKISKQLATILREIPLEISLEDLKVKEPNYEEVAKLFLRLEFKSFLKEIEPKIKNEYQESKELVQFEPVATEERIAVIFNNGFYVDDGEKTKFYSLEQLFEIQEIFRNKKIITDDAKGIYHVCLEKGLTFPEICFDARIAAYVLNPADQNPGLKGLYLKYGLPVFEDFSLNIRGLFYLWKEMMEKVHEQEQERLFYEIELPLTPVLAQMEHTGIQVDREALKEMSVELGEQIEELIREIYALAGEEFNLNSPRQLGVILFEKLGLPVIKKTKTGYSTDAEVLEELLPYHEIIGKILTYRQLMKLKSTYTDGLMPLINERTGKLHTTFNQTGTLTGRLASSEPNLQNIPVRLELGRKLRKMFIPSPGYDYIVSADYSQIELRLLAHFSEEPKLIEAYQKGEDIHRKTASEVFGVPLEEVTPEMRAHAKSVNFGIVYGISDFGLGRDLKIPREIAGKYIKNYFTNYPRVREYLDELVRTAREKGYVTTLFGRRRYIPELSSKNRTVQGFGERTAMNTPLQGSAADIIKLAMINVERELKNSKLRSRLLLSVHDELVLEVPAEELEEVKGLVKGVMESVVELKVPLIAEVGAGKNWYEAK

SEQ ID NO: 4 - protein, 586 aa, Carboxydothermus pertinax
MGIPLEISLEDLKVKEPDYEEAAKLFTRLEFKSFLKEVEPKVKKEYQESKETVNIEIIKAEGQVVVVFNDGFYVDDGEKTSFYSLDQLVDLQEIFRGKEIITDDAKGIYRFCLEKGISFPKVNFDARIAAYVLNPADQNPGLNGLYIKYNLPVYDDLFLNIRGLFYLKKEMLAKIREQQQEKLYQEIELPLTPVLARMEFTGIQVDREALKEMSLELGEQMEALTREIYSLAGEEFNLNSPKQLGVILFEKLGLPVIKKTKTGYSTDAEVLEELFAYHEIVGKILNYRQLMKLKSTYTDGLMPLINEYTGKLHTTFNQTGTLTGRLASSEPNLQNIPVRLELGRKLRKMFIPSTGYDYIISADYSQIELRLLAHFSEEPKLIEAYQKGEDIHRKTAAEVFGVPLEEVSLEMRSHAKSVNFGIVYGISDFGLGRDLKIPREVAGKYIKNYFANYPKVREYLDGLIRTAREKGYVTTLFGRRRYIPELTAKNRTVQSFGERTAMNTPLQGTAADIIKLAMINMERELLRKGLKSRLLLSVHDELVLEVPAEEVEEVKSLVKKVMESVVELKVPLIAEVGMGKNWYEAK

SEQ ID NO: 5 - protein, 831 aa, Carboxydothermus pertinax
MGKVVLVDGNSLLHRAFFALPPLKTSKGEPTGAVYGFLTMLFKVIKDENPDYLAVAFDVSRKTFRSDQFSAYKGHRKEAPEELVPQFALVREVLKVLNIPYIELMGYEADDIIGHLSKVFVNHGHEVKIYTADRDMLQLVEEKVAVYLTKKGITDLVKMDLAKVQENYGLKPIQLIDVKGLMGDPSDNIPGVPGIGEKTALDLIKTYGSLEEVLTHKEELKPKLKEKLTEHENLAKISKELATILREIPLEISLEDLKVKEPDYEEAAKLFTRLEFKSFLKEVEPKVKKEYQESKETVNIEIIKAEGQVVVVFNDGFYVDDGEKTSFYSLDQLVDLQEIFRGKEIITDDAKGIYRFCLEKGISFPKVNFDARIAAYVLNPADQNPGLNGLYIKYNLPVYDDLFLNIRGLFYLKKEMLAKIREQQQEKLYQEIELPLTPVLARMEFTGIQVDREALKEMSLELGEQMEALTREIYSLAGEEFNLNSPKQLGVILFEKLGLPVIKKTKTGYSTDAEVLEELFAYHEIVGKILNYRQLMKLKSTYTDGLMPLINEYTGKLHTTFNQTGTLTGRLASSEPNLQNIPVRLELGRKLRKMFIPSTGYDYIISADYSQIELRLLAHFSEEPKLIEAYQKGEDIHRKTAAEVFGVPLEEVSLEMRSHAKSVNYGIVYGISDFGLGRDLKIPREVAGKYIKNYFANYPKVREYLDGLIRTAREKGYVTTLFGRRRYIPELTAKNRTVQSFGERTAMNTPLQGTAADIIKLAMINMERELLRKGLKSRLLLSVHDELVLEVPAEEVEEVKSLVKKVMESVVELKVPLIAEVGMGKNWYEAK

SEQ ID NO: 6 - protein, 831 aa, Carboxydothermus ferrireducens
MGKVVLVDGNSLLHRAFFALPPLKTTKGEPTGAVYGFLTMLFRVIKDEKPEYLAVAFDVSRKTFRTKQFTAYKGHRKEAPDELVPQFALVREVLKVLNVPYIELDGYEADDIIGHLSRVFAGQGHEVVIYTADRDMLQLVDEKTVVYLTKKGITELVKMDLAAILESYGLKPKQLVDVKGLMGDPSDNIPGVPGIGEKTALDLIKTYGSVEEVLAHKDELKPKLREKLAEHENLAKISKQLATILREIPLEISLEDLKVKEPNYEEVAKLFLRLEFKSFLKEIEPKIKKEYQEGKDLVQFETVETEGQIAVVFSDGFYVDDGEKTKFYSLDRLNEIQEIFRNKKIITDDAKGIYHVCLEKGLTFPEVCFDARIAAYVLNPADQNPGLKGLYLKYDLPVYEDVSLNIRGLFYLKKEMMKKIFEQEQERLFYEIELPLTPVLAQMEHTGIQVDREALKEMSLELGEQIEELIREIYALAGEEFNLNSPKQLGVILFEKLGLPVIKKTKTGYSTDAEVLEELLPYHEIIGKILNYRQLMKLKSTYTDGLMPLINERTGKLHTTFNQTGTLTGRLASSEPNLQNIPIRLELGRKLRKMFIPSPGYDYIVSADYSQIELRLLAHFSEEPKLIEAYQKGEDIHRKTASEVFGVSLEEVTPEMRAHAKSVNYGIVYGISDFGLGRDLKIPREVAGKYIKNYFANYPKVREYLDELVRTAREKGYVTTLFGRRRYIPELSSKNRTVQGFGERTAMNTPLQGSAADIIKLAMINVEKELKARKLKSRLLLSVHDELVLEVPAEELEEVKALVKGVMESVVELKVPLIAEVGAGKNWYEAK

SEQ ID NO: 7 - protein, 831 aa, Carboxydothermus islandicus
MGKVVLVDGNSLLHRAFFALPPLKTTKGEPTGAVYGFLTMLFRVIRDEKPEYLAVAFDVSRKTFRTEQFTAYKGHRKEAPDELVPQFALVREVLKVLNVPYIELDGYEADDIIGHLSRVFAGQGHEVVIYTADRDMLQLVDEKTVVYLTKKGITELVKMDLAEVLENYGLKPQQLVDVKGLMGDPSDNIPGVPGIGEKTALDLIKTYGSVEEVLARKEELKPKLREKLAEHESLAKISKQLATILREIPLEISLEDLKVKEPNYEEVAKLFLRLEFKSFLKEIEPKIKNEYQESKELVQFEPVATEERIAVIFNNGFYVDDGEKTKFYSLEQLFEIQEIFRNKKIITDDAKGIYHVCLEKGLTFPEICFDARIAAYVLNPADQNPGLKGLYLKYGLPVFEDFSLNIRGLFYLWKEMMEKVHEQEQERLFYEIELPLTPVLAQMEHTGIQVDREALKEMSVELGEQIEELIREIYALAGEEFNLNSPRQLGVILFEKLGLPVIKKTKTGYSTDAEVLEELLPYHEIIGKILTYRQLMKLKSTYTDGLMPLINERTGKLHTTFNQTGTLTGRLASSEPNLQNIPVRLELGRKLRKMFIPSPGYDYIVSADYSQIELRLLAHFSEEPKLIEAYQKGEDIHRKTASEVFGVPLEEVTPEMRAHAKSVNYGIVYGISDFGLGRDLKIPREIAGKYIKNYFTNYPRVREYLDELVRTAREKGYVTTLFGRRRYIPELSSKNRTVQGFGERTAMNTPLQGSAADIIKLAMINVERELKNSKLRSRLLLSVHDELVLEVPAEELEEVKGLVKGVMESVVELKVPLIAEVGAGKNWYEAK

SEQ ID NO: 8 - protein, 586 aa, Carboxydothermus pertinax
MGIPLEISLEDLKVKEPDYEEAAKLFTRLEFKSFLKEVEPKVKKEYQESKETVNIEIIKAEGQVVVVFNDGFYVDDGEKTSFYSLDQLVDLQEIFRGKEIITDDAKGIYRFCLEKGISFPKVNFDARIAAYVLNPADQNPGLNGLYIKYNLPVYDDLFLNIRGLFYLKKEMLAKIREQQQEKLYQEIELPLTPVLARMEFTGIQVDREALKEMSLELGEQMEALTREIYSLAGEEFNLNSPKQLGVILFEKLGLPVIKKTKTGYSTDAEVLEELFAYHEIVGKILNYRQLMKLKSTYTDGLMPLINEYTGKLHTTFNQTGTLTGRLASSEPNLQNIPVRLELGRKLRKMFIPSTGYDYIISADYSQIELRLLAHFSEEPKLIEAYQKGEDIHRKTAAEVFGVPLEEVSLEMRSHAKSVNYGIVYGISDFGLGRDLKIPREVAGKYIKNYFANYPKVREYLDGLIRTAREKGYVTTLFGRRRYIPELTAKNRTVQSFGERTAMNTPLQGTAADIIKLAMINMERELLRKGLKSRLLLSVHDELVLEVPAEEVEEVKSLVKKVMESVVELKVPLIAEVGMGKNWYEAK

SEQ ID NO: 9 - DNA, 2496 bp, Carboxydothermus pertinax
  source 1..2496: mol_type = genomic DNA
atgggaaaagtagttttggttgatggaaatagtttattacatagagccttttttgccctgccgcccctaaaaacttccaaaggtgagcctaccggggcggtttacgggtttttaaccatgctttttaaagtcataaaagacgaaaatcccgattatctcgcggtggcctttgatgttagccggaagactttccgcagcgaccagttttcagcttataaaggacaccgtaaggaagctcccgaggaactggtacctcaatttgccctggtgcgggaggtattaaaggttttaaatattccctatatagagcttatggggtacgaagctgatgatattatcggccatttatcaaaggtttttgtgaaccatggccatgaggtaaaaatctatacggctgaccgggatatgttacaattggtagaggaaaaagtggcagtttaccttacgaagaaagggataaccgatcttgtcaaaatggatttagctaaagtgcaagaaaactatggcttaaaaccgatccagcttattgatgttaaaggcttaatgggagacccttcggataatataccgggagttcctggaattggtgagaagaccgctttggatttaatcaaaacttatggttcattagaagaagttttgacccataaagaggagttgaaacccaaattaaaagaaaagcttaccgaacacgaaaatttagcaaaaatttctaaagaattagctacaattctgcgggagataccgttagaaatttccttggaggatttaaaagtcaaagaaccagattatgaagaagcggctaaactttttacccgtttagagtttaaaagcttcttaaaagaggtagaacccaaagtaaaaaaagaataccaagaaagcaaagagacggtgaatattgaaattataaaggctgaaggccaagtagttgtggtctttaatgatggattttatgttgatgatggagaaaagacaagtttttactctttagaccaattggttgatttgcaagaaatcttccggggaaaagaaatcataaccgatgatgccaaaggaatttaccgcttttgtctggaaaaaggtatttcttttcctaaagtgaattttgatgcaagaattgcagcgtatgtattaaatcctgccgaccaaaatccggggcttaacggactgtatataaaatataatttaccggtgtatgacgacctttttttaaacattagaggtttattttatctaaaaaaagaaatgctggcgaaaatacgggaacagcagcaggaaaagttatatcaagaaattgaacttcctttaactccggtcctagcccggatggagtttaccggcattcaggtggatcgggaagctttaaaagagatgtcgttagagcttggggagcaaatggaagcgttaacccgggaaatctattccctggcgggagaagagtttaatttaaactcgcccaagcaattaggggttatcctctttgaaaaattaggtctccccgtaattaagaagacgaaaaccggctactctaccgatgcggaagtattagaagagctttttgcgtatcatgaaattgtagggaaaatattaaattaccggcagcttatgaagttaaagtctacctataccgatggtttaatgcctttaatcaatgagtacaccggtaaacttcatactacttttaatcaaacaggcactttaaccggacgtctggcctcctcggagcccaatctccaaaatatccctgtgcggctcgagcttgggcgtaaattgcgcaaaatgtttattccttctacgggttatgattatataatctcggcggattattcacaaattgagctaaggttacttgctcacttttccgaagaaccaaagttaatcgaagcgtaccaaaaaggagaagatattcatcggaaaactgccgcggaagtgtttggggtacctttagaggaagtatctttagaaatgcggtctcatgctaaatcggtaaattttggtattgtatacggaattagtgactttgggcttggcagggatttaaaaattccccgggaggttgccgggaaatacattaaaaactattttgccaactatccaaaggttcgggagtatttagatgggcttattcggactgctagagaaaaggggtatgtgaccactttatttggacgaaggcggtatattccggagttaaccgctaaaaatcgtacagtacagagctttggtgagcggactgccatgaatacgccgctccagggtactgcggctgatataattaaacttgcaatgattaacatggagagagagcttttacggaaggggttaaaatcccggttgttactttcggtgcacgacgaacttgttttagaggtaccggcagaggaagtggaggaagtaaaatccctggtaaaaaaggttatggaatcagtggtggaactaaaagttcccctgattgctgaagttggcatggggaaaaactggtatgaagctaaataa

SEQ ID NO: 10 - DNA, 2496 bp, Carboxydothermus ferrireducens
  source 1..2496: mol_type = genomic DNA
atgggaaaagtagtcctggtggatggaaatagtttattacatagagcgttttttgcccttccgcccttaaaaactactaaaggagagcctaccggggcggtttacgggtttttaacgatgctttttcgggtaataaaagatgaaaaacccgaatatttagcggtagcctttgatgttagccggaaaacctttcgcaccaagcagtttactgcatacaaagggcaccgcaaagaagccccggatgagcttgtaccccagtttgccctggtgcgggaagtattaaaggttttaaatgtgccctatattgaacttgacggttatgaggccgatgatattatcggccacctatcaagggtttttgcggggcaaggacatgaagtggtgatttataccgctgaccgggacatgctgcaactggtagatgaaaaaacggtggtataccttaccaaaaaaggcattaccgaactggttaaaatggatttagctgcgattttagaaagctacggcttaaaacccaaacagcttgtggatgttaaaggattaatgggagatccctcggacaacatacccggggttcccgggattggggagaaaactgctttagatttaattaaaacttatggctcagtggaagaagttttggcccataaagatgagttaaaacctaaattaagagaaaagcttgccgaacacgaaaatttagcaaaaatatcgaaacaattagctacaatcctgcgggaaataccgttagaaatctccctggaagatttaaaagtgaaagaacctaattatgaagaagttgctaaattatttcttcgccttgagtttaaaagctttttaaaagaaatagaaccaaaaataaagaaagaataccaggaaggtaaagatttggtgcaatttgaaactgtagaaacggaaggacagattgcagtagtttttagtgatggattttacgttgatgacggggaaaaaacaaagttttactcgttagaccggctgaatgaaatacaggaaatatttaggaataaaaaaattattaccgacgatgccaaaggaatttatcatgtctgtttagaaaaaggtctgacttttcccgaagtttgttttgatgcgcggattgcagcttatgttttaaacccggccgaccaaaatcccggcctcaaggggctttatctaaagtatgacttaccggtgtatgaagatgtatctttaaacattagagggttgttttatttaaaaaaagaaatgatgaaaaaaatctttgagcaggagcaggaaaggttattttatgaaatagaacttcctttaactccagttcttgctcaaatggagcataccggcattcaggttgaccgggaagctttaaaagagatgtcgttggagctgggagagcaaattgaagagttaatccgggaaatttatgcgctggcgggggaagagtttaacttaaattcgcccaagcagctgggagttattctttttgaaaaacttgggctgccggtaattaaaaagaccaagacgggctactctaccgatgcggaggttttggaagagctcttgccttaccacgaaattatcggcaaaatattgaattaccggcagcttatgaagttaaaatccacttatactgacggcttaatgccattaataaatgagcgtaccggtaaacttcacactacttttaaccagaccggtactttaaccggacgcctggcgtcttcggagcccaatctccaaaatattcccatccggttggaactcggtcggaaattacgcaagatgtttataccttcaccggggtatgattatattgtttcggcggattattcccagattgaattaaggcttcttgcccatttttccgaagagcccaagcttattgaagcttaccaaaaaggggaggatattcaccggaaaacagcctccgaggtgttcggtgtatctttggaagaagttactcccgagatgcgcgctcatgccaagtcggtgaacttcggcattgtttatggcattagtgattttggtttaggcagagacttaaagattccccgggaggttgccggtaagtacattaaaaattattttgccaactatcccaaagtgcgggagtatctcgatgaacttgtccgtacggcaagagaaaagggttatgtgaccactttatttgggcgaagacgctatattcctgagctatcttcaaaaaaccgcacggttcaggggtttggcgaaaggacggccatgaatactccccttcagggctcggctgccgatattattaagcttgcaatgattaatgtagaaaaagaacttaaagcccgtaagcttaagtcccggctccttctttcggtgcacgatgagttagttttagaagtgccggcggaagagctggaagaggtaaaagcgctggtaaaaggggttatggagtcggtggttgaactgaaagtgcctttaatcgctgaagttggtgcaggcaaaaactggtatgaagcgaagtaa

SEQ ID NO: 11 - DNA, 2496 bp, Carboxydothermus islandicus
  source 1..2496: mol_type = genomic DNA
atgggaaaagtagtcctggtggatggaaatagtttattacatagagcgttttttgcccttccgcctttaaaaactactaaaggggagcctaccggggcggtttacgggtttttaacgatgctttttcgggtaataagagatgaaaaaccagaatatttagcggtagcttttgatgttagccggaaaacctttcgtaccgagcagtttactgcatacaaagggcaccgcaaagaagctccggatgaactggtaccccagtttgccctggtacgggaagtattaaaagttttaaatgtgccctatattgaacttgacggctatgaggcagatgatattatcggccacctatcaagggtgtttgccgggcaggggcatgaggtagtaatttacaccgctgaccgggacatgctgcaactggtcgatgaaaaaaccgtggtataccttaccaaaaaaggcattaccgaactggttaaaatggatttagctgaggttttggaaaactacggcttaaaacctcagcagcttgtggatgttaaaggattaatgggtgatccctcggacaacatacccggagttccggggattggggagaaaactgctttagatttaataaaaacttatggctcggtggaagaagttttggcccgcaaagaagagttaaaacctaaattaagagaaaaacttgccgaacatgaaagtttagcgaaaatatcgaaacaattagctactatcctgcgggaaataccgttagaaatctccctggaagatttaaaagtaaaagaacctaattatgaagaagtagctaaattatttcttcgccttgagtttaaaagctttttaaaagaaatagaaccaaaaataaagaatgaataccaggaaagtaaagagttggtgcaatttgaaccggtagcaacggaagaacggattgcggtaatctttaataatggattttatgttgatgacggggaaaaaacaaagttttattctttagaacagctttttgaaatacaggaaatatttcggaataaaaaaattattaccgacgacgccaaaggaatttatcatgtctgtttagaaaaaggtctgacttttcccgaaatttgttttgatgcgcggatagcagcttatgttttaaatccggccgaccaaaatcccgggctcaaggggctgtatttaaagtatggtttgccggtgtttgaagatttttctttaaatatcaggggtttattttatttatggaaagaaatgatggaaaaagttcacgagcaggagcaggaaaggttattttatgaaatagaacttcctctaactccggttcttgctcaaatggagcataccggcattcaggttgaccgggaagctttaaaagaaatgtcggtggagctgggagagcaaattgaagagttaatccgggaaatttatgcgctggcgggggaagagtttaacttaaactctcccaggcagctgggagttattctttttgaaaaacttgggctgccggtaattaaaaagaccaagacgggctactctaccgatgcggaggttttggaagagcttttaccttaccacgaaattattggcaaaattttgacttaccgtcagcttatgaagttaaaatcaacctataccgacggtttaatgcctttaataaatgagcgtaccggtaaacttcacaccacttttaaccagaccggtaccttaaccggacgtctggcgtcttcggagcccaatcttcaaaatattccggtgcggttggaactcggccggaaattacgcaagatgtttataccttcccctggttatgattatattgtttcggcggattattcccagattgagttaaggcttcttgctcatttttccgaagagcccaagcttattgaagcgtaccaaaaaggggaggatattcaccggaaaaccgcttcagaagtatttggtgtacctttagaagaagttacccctgagatgcgtgctcatgccaagtcggtgaacttcggcattgtctatggcattagcgattttggtttaggcagagatttaaagattccccgggagattgccggaaaatatattaaaaattattttaccaattatcccagagtgcgggagtatctcgatgaacttgtccgtacggcaagagaaaagggatatgtgaccacgttatttgggcgaagacgttatattccggagctatcgtccaaaaatcgcacagtgcagggttttggcgaaaggacggccatgaataccccccttcagggctcagctgccgatattattaagcttgcaatgattaatgtagaaagggaattaaaaaacagcaagcttaggtcccggctccttctttcggtgcacgatgagttagttttggaagtgccggcggaagagctggaagaagttaaagggctggtaaaaggggttatggagtcggtggttgaactaaaagtgcctttaatcgctgaagttggcgcaggaaaaaactggtatgaagccaagtaa

SEQ ID NO: 12 - DNA, 1761 bp, Carboxydothermus pertinax
  source 1..1761: mol_type = genomic DNA
atgggcataccgttagaaatttccttggaggatttaaaagtcaaagaaccagattatgaagaagcggctaaactttttacccgtttagagtttaaaagcttcttaaaagaggtagaacccaaagtaaaaaaagaataccaagaaagcaaagagacggtgaatattgaaattataaaggctgaaggccaagtagttgtggtctttaatgatggattttatgttgatgatggagaaaagacaagtttttactctttagaccaattggttgatttgcaagaaatcttccggggaaaagaaatcataaccgatgatgccaaaggaatttaccgcttttgtctggaaaaaggtatttcttttcctaaagtgaattttgatgcaagaattgcagcgtatgtattaaatcctgccgaccaaaatccggggcttaacggactgtatataaaatataatttaccggtgtatgacgacctttttttaaacattagaggtttattttatctaaaaaaagaaatgctggcgaaaatacgggaacagcagcaggaaaagttatatcaagaaattgaacttcctttaactccggtcctagcccggatggagtttaccggcattcaggtggatcgggaagctttaaaagagatgtcgttagagcttggggagcaaatggaagcgttaacccgggaaatctattccctggcgggagaagagtttaatttaaactcgcccaagcaattaggggttatcctctttgaaaaattaggtctccccgtaattaagaagacgaaaaccggctactctaccgatgcggaagtattagaagagctttttgcgtatcatgaaattgtagggaaaatattaaattaccggcagcttatgaagttaaagtctacctataccgatggtttaatgcctttaatcaatgagtacaccggtaaacttcatactacttttaatcaaacaggcactttaaccggacgtctggcctcctcggagcccaatctccaaaatatccctgtgcggctcgagcttgggcgtaaattgcgcaaaatgtttattccttctacgggttatgattatataatctcggcggattattcacaaattgagctaaggttacttgctcacttttccgaagaaccaaagttaatcgaagcgtaccaaaaaggagaagatattcatcggaaaactgccgcggaagtgtttggggtacctttagaggaagtatctttagaaatgcggtctcatgctaaatcggtaaattttggtattgtatacggaattagtgactttgggcttggcagggatttaaaaattccccgggaggttgccgggaaatacattaaaaactattttgccaactatccaaaggttcgggagtatttagatgggcttattcggactgctagagaaaaggggtatgtgaccactttatttggacgaaggcggtatattccggagttaaccgctaaaaatcgtacagtacagagctttggtgagcggactgccatgaatacgccgctccagggtactgcggctgatataattaaacttgcaatgattaacatggagagagagcttttacggaaggggttaaaatcccggttgttactttcggtgcacgacgaacttgttttagaggtaccggcagaggaagtggaggaagtaaaatccctggtaaaaaaggttatggaatcagtggtggaactaaaagttcccctgattgctgaagttggcatggggaaaaactggtatgaagctaaataa

SEQ ID NO: 13 - DNA, 2496 bp, Carboxydothermus pertinax
  source 1..2496: mol_type = genomic DNA
atgggaaaagtagttttggttgatggaaatagtttattacatagagccttttttgccctgccgcccctaaaaacttccaaaggtgagcctaccggggcggtttacgggtttttaaccatgctttttaaagtcataaaagacgaaaatcccgattatctcgcggtggcctttgatgttagccggaagactttccgcagcgaccagttttcagcttataaaggacaccgtaaggaagctcccgaggaactggtacctcaatttgccctggtgcgggaggtattaaaggttttaaatattccctatatagagcttatggggtacgaagctgatgatattatcggccatttatcaaaggtttttgtgaaccatggccatgaggtaaaaatctatacggctgaccgggatatgttacaattggtagaggaaaaagtggcagtttaccttacgaagaaagggataaccgatcttgtcaaaatggatttagctaaagtgcaagaaaactatggcttaaaaccgatccagcttattgatgttaaaggcttaatgggagacccttcggataatataccgggagttcctggaattggtgagaagaccgctttggatttaatcaaaacttatggttcattagaagaagttttgacccataaagaggagttgaaacccaaattaaaagaaaagcttaccgaacacgaaaatttagcaaaaatttctaaagaattagctacaattctgcgggagataccgttagaaatttccttggaggatttaaaagtcaaagaaccagattatgaagaagcggctaaactttttacccgtttagagtttaaaagcttcttaaaagaggtagaacccaaagtaaaaaaagaataccaagaaagcaaagagacggtgaatattgaaattataaaggctgaaggccaagtagttgtggtctttaatgatggattttatgttgatgatggagaaaagacaagtttttactctttagaccaattggttgatttgcaagaaatcttccggggaaaagaaatcataaccgatgatgccaaaggaatttaccgcttttgtctggaaaaaggtatttcttttcctaaagtgaattttgatgcaagaattgcagcgtatgtattaaatcctgccgaccaaaatccggggcttaacggactgtatataaaatataatttaccggtgtatgacgacctttttttaaacattagaggtttattttatctaaaaaaagaaatgctggcgaaaatacgggaacagcagcaggaaaagttatatcaagaaattgaacttcctttaactccggtcctagcccggatggagtttaccggcattcaggtggatcgggaagctttaaaagagatgtcgttagagcttggggagcaaatggaagcgttaacccgggaaatctattccctggcgggagaagagtttaatttaaactcgcccaagcaattaggggttatcctctttgaaaaattaggtctccccgtaattaagaagacgaaaaccggctactctaccgatgcggaagtattagaagagctttttgcgtatcatgaaattgtagggaaaatattaaattaccggcagcttatgaagttaaagtctacctataccgatggtttaatgcctttaatcaatgagtacaccggtaaacttcatactacttttaatcaaacaggcactttaaccggacgtctggcctcctcggagcccaatctccaaaatatccctgtgcggctcgagcttgggcgtaaattgcgcaaaatgtttattccttctacgggttatgattatataatctcggcggattattcacaaattgagctaaggttacttgctcacttttccgaagaaccaaagttaatcgaagcgtaccaaaaaggagaagatattcatcggaaaactgccgcggaagtgtttggggtacctttagaggaagtatctttagaaatgcggtctcatgctaaatcggtaaattatggtattgtatacggaattagtgactttgggcttggcagggatttaaaaattccccgggaggttgccgggaaatacattaaaaactattttgccaactatccaaaggttcgggagtatttagatgggcttattcggactgctagagaaaaggggtatgtgaccactttatttggacgaaggcggtatattccggagttaaccgctaaaaatcgtacagtacagagctttggtgagcggactgccatgaatacgccgctccagggtactgcggctgatataattaaacttgcaatgattaacatggagagagagcttttacggaaggggttaaaatcccggttgttactttcggtgcacgacgaacttgttttagaggtaccggcagaggaagtggaggaagtaaaatccctggtaaaaaaggttatggaatcagtggtggaactaaaagttcccctgattgctgaagttggcatggggaaaaactggtatgaagctaaataa

SEQ ID NO: 14 - DNA, 2496 bp, Carboxydothermus ferrireducens
  source 1..2496: mol_type = genomic DNA
atgggaaaagtagtcctggtggatggaaatagtttattacatagagcgttttttgcccttccgcccttaaaaactactaaaggagagcctaccggggcggtttacgggtttttaacgatgctttttcgggtaataaaagatgaaaaacccgaatatttagcggtagcctttgatgttagccggaaaacctttcgcaccaagcagtttactgcatacaaagggcaccgcaaagaagccccggatgagcttgtaccccagtttgccctggtgcgggaagtattaaaggttttaaatgtgccctatattgaacttgacggttatgaggccgatgatattatcggccacctatcaagggtttttgcggggcaaggacatgaagtggtgatttataccgctgaccgggacatgctgcaactggtagatgaaaaaacggtggtataccttaccaaaaaaggcattaccgaactggttaaaatggatttagctgcgattttagaaagctacggcttaaaacccaaacagcttgtggatgttaaaggattaatgggagatccctcggacaacatacccggggttcccgggattggggagaaaactgctttagatttaattaaaacttatggctcagtggaagaagttttggcccataaagatgagttaaaacctaaattaagagaaaagcttgccgaacacgaaaatttagcaaaaatatcgaaacaattagctacaatcctgcgggaaataccgttagaaatctccctggaagatttaaaagtgaaagaacctaattatgaagaagttgctaaattatttcttcgccttgagtttaaaagctttttaaaagaaatagaaccaaaaataaagaaagaataccaggaaggtaaagatttggtgcaatttgaaactgtagaaacggaaggacagattgcagtagtttttagtgatggattttacgttgatgacggggaaaaaacaaagttttactcgttagaccggctgaatgaaatacaggaaatatttaggaataaaaaaattattaccgacgatgccaaaggaatttatcatgtctgtttagaaaaaggtctgacttttcccgaagtttgttttgatgcgcggattgcagcttatgttttaaacccggccgaccaaaatcccggcctcaaggggctttatctaaagtatgacttaccggtgtatgaagatgtatctttaaacattagagggttgttttatttaaaaaaagaaatgatgaaaaaaatctttgagcaggagcaggaaaggttattttatgaaatagaacttcctttaactccagttcttgctcaaatggagcataccggcattcaggttgaccgggaagctttaaaagagatgtcgttggagctgggagagcaaattgaagagttaatccgggaaatttatgcgctggcgggggaagagtttaacttaaattcgcccaagcagctgggagttattctttttgaaaaacttgggctgccggtaattaaaaagaccaagacgggctactctaccgatgcggaggttttggaagagctcttgccttaccacgaaattatcggcaaaatattgaattaccggcagcttatgaagttaaaatccacttatactgacggcttaatgccattaataaatgagcgtaccggtaaacttcacactacttttaaccagaccggtactttaaccggacgcctggcgtcttcggagcccaatctccaaaatattcccatccggttggaactcggtcggaaattacgcaagatgtttataccttcaccggggtatgattatattgtttcggcggattattcccagattgaattaaggcttcttgcccatttttccgaagagcccaagcttattgaagcttaccaaaaaggggaggatattcaccggaaaacagcctccgaggtgttcggtgtatctttggaagaagttactcccgagatgcgcgctcatgccaagtcggtgaactacggcattgtttatggcattagtgattttggtttaggcagagacttaaagattccccgggaggttgccggtaagtacattaaaaattattttgccaactatcccaaagtgcgggagtatctcgatgaacttgtccgtacggcaagagaaaagggttatgtgaccactttatttgggcgaagacgctatattcctgagctatcttcaaaaaaccgcacggttcaggggtttggcgaaaggacggccatgaatactccccttcagggctcggctgccgatattattaagcttgcaatgattaatgtagaaaaagaacttaaagcccgtaagcttaagtcccggctccttctttcggtgcacgatgagttagttttagaagtgccggcggaagagctggaagaggtaaaagcgctggtaaaaggggttatggagtcggtggttgaactgaaagtgcctttaatcgctgaagttggtgcaggcaaaaactggtatgaagcgaagtaa

SEQ ID NO: 15 - DNA, 2496 bp, Carboxydothermus islandicus
  source 1..2496: mol_type = genomic DNA
atgggaaaagtagtcctggtggatggaaatagtttattacatagagcgttttttgcccttccgcctttaaaaactactaaaggggagcctaccggggcggtttacgggtttttaacgatgctttttcgggtaataagagatgaaaaaccagaatatttagcggtagcttttgatgttagccggaaaacctttcgtaccgagcagtttactgcatacaaagggcaccgcaaagaagctccggatgaactggtaccccagtttgccctggtacgggaagtattaaaagttttaaatgtgccctatattgaacttgacggctatgaggcagatgatattatcggccacctatcaagggtgtttgccgggcaggggcatgaggtagtaatttacaccgctgaccgggacatgctgcaactggtcgatgaaaaaaccgtggtataccttaccaaaaaaggcattaccgaactggttaaaatggatttagctgaggttttggaaaactacggcttaaaacctcagcagcttgtggatgttaaaggattaatgggtgatccctcggacaacatacccggagttccggggattggggagaaaactgctttagatttaataaaaacttatggctcggtggaagaagttttggcccgcaaagaagagttaaaacctaaattaagagaaaaacttgccgaacatgaaagtttagcgaaaatatcgaaacaattagctactatcctgcgggaaataccgttagaaatctccctggaagatttaaaagtaaaagaacctaattatgaagaagtagctaaattatttcttcgccttgagtttaaaagctttttaaaagaaatagaaccaaaaataaagaatgaataccaggaaagtaaagagttggtgcaatttgaaccggtagcaacggaagaacggattgcggtaatctttaataatggattttatgttgatgacggggaaaaaacaaagttttattctttagaacagctttttgaaatacaggaaatatttcggaataaaaaaattattaccgacgacgccaaaggaatttatcatgtctgtttagaaaaaggtctgacttttcccgaaatttgttttgatgcgcggatagcagcttatgttttaaatccggccgaccaaaatcccgggctcaaggggctgtatttaaagtatggtttgccggtgtttgaagatttttctttaaatatcaggggtttattttatttatggaaagaaatgatggaaaaagttcacgagcaggagcaggaaaggttattttatgaaatagaacttcctctaactccggttcttgctcaaatggagcataccggcattcaggttgaccgggaagctttaaaagaaatgtcggtggagctgggagagcaaattgaagagttaatccgggaaatttatgcgctggcgggggaagagtttaacttaaactctcccaggcagctgggagttattctttttgaaaaacttgggctgccggtaattaaaaagaccaagacgggctactctaccgatgcggaggttttggaagagcttttaccttaccacgaaattattggcaaaattttgacttaccgtcagcttatgaagttaaaatcaacctataccgacggtttaatgcctttaataaatgagcgtaccggtaaacttcacaccacttttaaccagaccggtaccttaaccggacgtctggcgtcttcggagcccaatcttcaaaatattccggtgcggttggaactcggccggaaattacgcaagatgtttataccttcccctggttatgattatattgtttcggcggattattcccagattgagttaaggcttcttgctcatttttccgaagagcccaagcttattgaagcgtaccaaaaaggggaggatattcaccggaaaaccgcttcagaagtatttggtgtacctttagaagaagttacccctgagatgcgtgctcatgccaagtcggtgaactacggcattgtctatggcattagcgattttggtttaggcagagatttaaagattccccgggagattgccggaaaatatattaaaaattattttaccaattatcccagagtgcgggagtatctcgatgaacttgtccgtacggcaagagaaaagggatatgtgaccacgttatttgggcgaagacgttatattccggagctatcgtccaaaaatcgcacagtgcagggttttggcgaaaggacggccatgaataccccccttcagggctcagctgccgatattattaagcttgcaatgattaatgtagaaagggaattaaaaaacagcaagcttaggtcccggctccttctttcggtgcacgatgagttagttttggaagtgccggcggaagagctggaagaagttaaagggctggtaaaaggggttatggagtcggtggttgaactaaaagtgcctttaatcgctgaagttggcgcaggaaaaaactggtatgaagccaagtaa

SEQ ID NO: 16 - DNA, 1761 bp, Carboxydothermus pertinax
  source 1..1761: mol_type = genomic DNA
atgggcataccgttagaaatttccttggaggatttaaaagtcaaagaaccagattatgaagaagcggctaaactttttacccgtttagagtttaaaagcttcttaaaagaggtagaacccaaagtaaaaaaagaataccaagaaagcaaagagacggtgaatattgaaattataaaggctgaaggccaagtagttgtggtctttaatgatggattttatgttgatgatggagaaaagacaagtttttactctttagaccaattggttgatttgcaagaaatcttccggggaaaagaaatcataaccgatgatgccaaaggaatttaccgcttttgtctggaaaaaggtatttcttttcctaaagtgaattttgatgcaagaattgcagcgtatgtattaaatcctgccgaccaaaatccggggcttaacggactgtatataaaatataatttaccggtgtatgacgacctttttttaaacattagaggtttattttatctaaaaaaagaaatgctggcgaaaatacgggaacagcagcaggaaaagttatatcaagaaattgaacttcctttaactccggtcctagcccggatggagtttaccggcattcaggtggatcgggaagctttaaaagagatgtcgttagagcttggggagcaaatggaagcgttaacccgggaaatctattccctggcgggagaagagtttaatttaaactcgcccaagcaattaggggttatcctctttgaaaaattaggtctccccgtaattaagaagacgaaaaccggctactctaccgatgcggaagtattagaagagctttttgcgtatcatgaaattgtagggaaaatattaaattaccggcagcttatgaagttaaagtctacctataccgatggtttaatgcctttaatcaatgagtacaccggtaaacttcatactacttttaatcaaacaggcactttaaccggacgtctggcctcctcggagcccaatctccaaaatatccctgtgcggctcgagcttgggcgtaaattgcgcaaaatgtttattccttctacgggttatgattatataatctcggcggattattcacaaattgagctaaggttacttgctcacttttccgaagaaccaaagttaatcgaagcgtaccaaaaaggagaagatattcatcggaaaactgccgcggaagtgtttggggtacctttagaggaagtatctttagaaatgcggtctcatgctaaatcggtaaattatggtattgtatacggaattagtgactttgggcttggcagggatttaaaaattccccgggaggttgccgggaaatacattaaaaactattttgccaactatccaaaggttcgggagtatttagatgggcttattcggactgctagagaaaaggggtatgtgaccactttatttggacgaaggcggtatattccggagttaaccgctaaaaatcgtacagtacagagctttggtgagcggactgccatgaatacgccgctccagggtactgcggctgatataattaaacttgcaatgattaacatggagagagagcttttacggaaggggttaaaatcccggttgttactttcggtgcacgacgaacttgttttagaggtaccggcagaggaagtggaggaagtaaaatccctggtaaaaaaggttatggaatcagtggtggaactaaaagttcccctgattgctgaagttggcatggggaaaaactggtatgaagctaaataa

SEQ ID NO: 17 - DNA, 25 bp, Homo sapiens
  source 1..25: mol_type = genomic DNA
caaccgcgagaagatgacccagatc

SEQ ID NO: 18 - DNA, 25 bp, Homo sapiens
  source 1..25: mol_type = genomic DNA
gcaacgtacatggctggggtgttga

SEQ ID NO: 19 - DNA, 25 bp, Homo sapiens
  source 1..25: mol_type = genomic DNA
caaccgcgagaagatgacccagatc

SEQ ID NO: 20 - DNA, 25 bp, Homo sapiens
  source 1..25: mol_type = genomic DNA
ttcccgctcggccgtggtggtgaag

SEQ ID NO: 21 - DNA, 25 bp, Homo sapiens
  source 1..25: mol_type = other DNA
caaccgcgagaagatgacccagatc

SEQ ID NO: 22 - DNA, 30 bp, Homo sapiens
  modified_base 30: mod_base = OTHER; note = n is a dideoxycytidine monophosphate
  source 1..30: mol_type = other DNA
gcaacgtacatggctggggtgttgaaggtn

SEQ ID NO: 23 - DNA, 25 bp, Homo sapiens
  source 1..25: mol_type = other DNA
caaccgcgagaagatgacccagatc

SEQ ID NO: 24 - DNA, 30 bp, Homo sapiens
  modified_base 30: mod_base = OTHER; note = n is a dideoxycytidine monophosphate
  source 1..30: mol_type = other DNA
acagtgtgggtgaccccgtcaccggagtcn

SEQ ID NO: 25 - DNA, 28 bp, COVID-19 virus
  modified_base 28: mod_base = OTHER; note = n is dideoxycytidine monophosphate
  source 1..28: mol_type = other DNA
gagggagccttgaatacaccaaaagatn

SEQ ID NO: 26 - DNA, 28 bp, COVID-19 virus
  modified_base 28: mod_base = OTHER; note = n is a dideoxycytidine monophosphate
  source 1..28: mol_type = other DNA
gttgtagcacgattgcagcattgttagn

SEQ ID NO: 27 - DNA, 28 bp, Homo sapiens
  modified_base 28: mod_base = OTHER; note = n is a dideoxycytidine monophosphate
  source 1..28: mol_type = other DNA
agaagatgacccagatcatgtttgagan

SEQ ID NO: 28 - DNA, 27 bp, Homo sapiens
  modified_base 27: mod_base = OTHER; note = n is a dideoxycytidine monophosphate
  source 1..27: mol_type = other DNA
ttacagggatagcacagcctggatagn

(19) United States
(12) Patent Application Publication
(10) Pub. No.: US 2026/0258466 A1
(43) Pub. Date: Sep. 3, 2026

(54) Thermostable purified reverse transcriptase and methods of use

(71) Applicants: Shaofeng Ding, Santa Fe Springs, CA (US); Qiang Liu, Rancho Cucamonga, CA (US)

(72) Inventors: Shaofeng Ding, Santa Fe Springs, CA (US); Qiang Liu, Rancho Cucamonga, CA (US)

(21) Appl. No.: 19/679,721

(22) Filed: May 17, 2026

Related U.S. Application Data
(62) Division of application No. 17/541,254, filed on Dec. 3, 2021.
(60) Provisional application No. 63/124,055, filed on Dec. 11, 2020.

(51) Int. Cl. C12P 19/34; C12N 9/12

(52) U.S. Cl. CPC C12P 19/34; C12N 9/1252; C12Y 207/07007

(57) ABSTRACT

Thermophilic purified Type A DNA-dependent DNA polymerases efficiently catalyze reverse transcription and RNA-dependent DNA pyrophosphorolysis using ribonucleic acid (RNA) templates, virtually also being reverse transcriptases. At optimal temperatures of 60° C. to 62° C., the thermophilic reverse transcriptases are particularly efficient for reverse transcription in RT-PCR and RT-PAP amplifications.

### CROSS-REFERENCE TO RELATED APPLICATIONS

This US divisional patent application claims priority from U.S. nonprovisional patent application Ser. No. 17/541,254 filed on Dec. 3, 2021 and further claims priority from U.S. provisional patent application No. 63/124,055 filed on Dec. 11, 2020.

### SEQUENCE LISTING

This instant application contains a Sequence Listing which is submitted in XML format via Patent Center and is hereby incorporated by reference in its entirety. Said XML file is named “sequencelisting_st26_RTase” was created on Sep. 3, 2024 and is 5300 bites in size.

### BACKGROUND

The present invention relates to the field of molecular biology and particularly to thermostable reverse transcription and applications to RT-PCR and RT-PAP amplifications using ribonucleic acid (RNA) templates.

### CLASSIFICATION

“Heat-loving” or thermostable microorganisms thrive at high temperatures ≥50° C. Many such microorganisms are archaea, though they can be bacteria such as Carboxydothermus pertinax. One classification sorts these organisms into three distinct subtypes according to their optimal growth temperatures: 1) thermophiles with optimal temperatures between 50° C. and 64° C., 2) extreme thermophiles with optimal growth temperatures between 65° C. and 80° C., and 3) hyperthermophiles with optimal growth temperatures above 80° C.

Accordingly, their thermostable DNA polymerases (i.e., DNA-dependent DNA polymerases and RNA-dependent DNA polymerases or reverse transcriptases) can also be classified into three subtypes based on their optimal reaction temperatures: 1) thermophilic DNA polymerases with optimal reaction temperatures between 50° C. and 64° C., 2) extreme thermophilic DNA polymerases with optimal reaction temperatures between 65° C. and 80° C., and 3) hyperthermophilic DNA polymerases with optimal reaction temperatures above 80° C.

Herein, examples of thermophilic DNA-dependent DNA polymerases were found functioning as thermophilic RNA-dependent DNA polymerases or reverse transcriptases as well are disclosed.

### DNA Polymerase

DNA polymerase refers to a polymerase characterized as catalyzing synthesis, polymerization or extension of deoxyribonucleic acids in the 5′ to 3′ direction. DNA polymerase requires a primer to initiate the synthesis of a DNA strand along a complementary nucleic acid template:

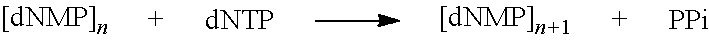

### DNA-Dependent DNA Polymerase

DNA-dependent DNA polymerase refers to a polymerase characterized as catalyzing DNA polymerization or extension using a DNA template.

Based on sequence homology with E. coli and other organisms, DNA-dependent DNA polymerases can be divided into six families: A, B, C, D, X and Y.

### RNA-Dependent DNA Polymerase or Reverse Transcriptase

Retroviruse can encode a special DNA polymerase called reverse transcriptase. It is characterized as catalyzing DNA polymerization or extension using an RNA template.

AMV and MMLV reverse transcriptases, commonly used mesophilic retroviral transcriptases at optimal temperatures of and 37° C., are isolated from Avian myeloblastosis virus (AMV) and moloney murine leukemia virus (MMLV) (Verma, 1977). However, at mesophilic temperatures, an RNA template keeps its secondary structures, resulting in premature termination of the reverse-transcribed DNA product.

Native Taq, a thermostable DNA-dependent DNA polymerase, is found to have measurable reverse transcriptase activity particularly in the presence of manganese ions (Jones and Foulkes, 1989). rTth, another thermostable DNA-dependent DNA polymerase, particularly in the presence of manganese ions, shows over 100-fold greater reverse transcriptase activity than Taq, even though they have no significant amino acid sequence similarity (Myers and Gelfand, 1991, Smith, et al., 2007). However, these DNA polymerases show low reverse transcriptase activities, leading to low efficiency of reverse transcription. In addition, they use Manganese ion, leading to low fidelity of reverse transcription.

### Pyrophorolysis

DNA polymerase can also catalyze pyrophosphorolysis, which is the reverse reaction of deoxyribonucleic acid (DNA) polymerization. In the presence of pyrophosphate, the 3′ nucleotide is removed by DNA polymerase from duplex DNA to generate a triphosphate nucleotide and a 3′ shortened duplex DNA (Deutscher and Kornberg, 1969):

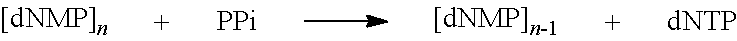

### DNA-Dependent DNA Pyrophosphorolysis

Taq, Tfl, TaqFS, Pfu and Vent DNA polymerases can catalyze DNA-dependent DNA pyrophosphorolysis using DNA templates (Liu and Sommer, 2000, Liu and Sommer, 2002, Liu and Sommer, 2004a, Gardner et al., 2004). HIV and HCV reverse transcriptases were also reported to catalyze DNA-dependent DNA pyrophosphorolysis that removes a dideoxynucleotide from a DNA primer on a synthetic DNA (rather than RNA) template (Arion, et al., 1998 and Urban, et al., 2001).

### RNA-Dependent DNA Pyrophosphorolysis

Besides DNA template, mutant Taq DNA polymerase having a F667Y mutation can catalyze RNA-dependent DNA pyrophosphorolysis that removes a dideoxyribonucleotide or acyclonucleotide from a primer using an RNA template. However, the efficiency is still relatively low (U.S. Pat. Nos. 9,133,491 and 9,926,543).

### Application to Nucleic Acid Amplification

### PCR and RT-PCR

PCR refers to polymerase chain reaction, a widely used method for nucleotide acid amplification. In PCR, DNA polymerase, such as Taq, amplifies from a DNA template exponentially to billions of copies.

RT-PCR refers to reverse-transcription PCR. RT-PCR includes two steps of 1) reverse transcription on an RNA template to produce cDNA by typically a non-thermostable viral reverse transcriptase, such as MMLV or AMV-RT, and 2) PCR amplification of the cDNA as template by a thermostable DNA-dependent DNA polymerase, such as Taq.

The current RT-PCR has three drawbacks. 1) At mesophilic temperatures: non-thermostable viral reverse transcriptases could not pass through an RNA template of stable secondary structures, resulting in premature termination. 2) Inhibition of downstream PCR: AMV and MMLV reverse transcriptases used in RT step inhibit the downstream PCR amplification, decreasing its efficiency. 3) Two step/tube procedure: due to incompatibility of reaction compositions, reverse transcription is conducted in a first tube and then PCR in a second tube, increasing potential contamination.

### PAP and RT-PAP (RNA-PAP)

Pyrophosphorolysis activated polymerization (PAP) is a method for nucleic acid amplification where pyrophosphorolysis and polymerization are serially coupled by DNA polymerase using 3′ blocked primers (Liu and Sommer, 2000). A primer is blocked at the 3′ end with a non-extendable nucleotide (3′ blocker), such as a dideoxynucleotide, and cannot be directly extended by DNA polymerase. When the 3′ blocked primer anneals to its complementary DNA template, DNA polymerase can remove the 3′ blocker from the 3′ blocked primer in the presence of pyrophosphate, which reaction is pyrophosphorolysis. The DNA polymerase can then extend the unblocked primer on the DNA template. In addition to references cited herein, PAP has been described in U.S. Pat. Nos. 6,534,269; 7,033,763; 7,105,298; 7,238,480; 7,504,221; 7,914,995 and 7,919,253.

Besides dideoxynucleotides, other 3′ blockers, such as acyclonucleotides that substitute a 2-hydroxyethoxymethyl group for the 2′-deoxyribofuranosyl sugar, are also used (Liu and Sommer, 2004a).

The serial coupling of pyrophosphorolysis and extension using the 3′ blocked primer in PAP results in an extremely high selectivity (Liu and Sommer, 2000, Liu and Sommer, 2004b) because a significant nonspecific amplification (false positive) requires mismatch pyrophosphorolysis followed by mis-incorporation by the DNA polymerase, an event with a frequency estimated to be 3.3×10-11.

### DNA-PAP

PAP was initially tested with Tfl and Taq DNA-dependent DNA polymerases using a DNA template of the human dopamine D1 gene, proving the principle that DNA-dependent DNA pyrophosphorolysis and DNA-dependent DNA polymerization can be serially coupled (Liu and Sommer, 2000) for amplification. The efficiency of PAP was greatly improved using TaqFS, a genetically engineered polymerase comprising a F667Y mutation, which were demonstrated using many other DNA templates (Liu and Sommer, 2002).

### RT-PAP (RNA-PAP)

RT-PAP was developed that can directly amplify from an RNA template. RT-PAP brings in a new mechanism for amplification of the RNA template in which RNA-dependent DNA pyrophosphorolysis removes a 3′ blocker, such as a dideoxynucleotide, from a 3′ blocked primer when hybridized to the RNA template, and then RNA-dependent DNA polymerization extends the unblocked primer (U.S. Pat. Nos. 9,133,491, 9,926,543). Due to the low efficiency, until now there has been no commercial use.

### Advantages of the Invention

RT-PCR: Thermostable purified DNA-dependent DNA polymerases efficiently catalyze reverse transcription on an RNA template at thermophilic temperatures, virtually also being reverse transcriptases or RNA-dependent DNA polymerases. Besides reverse transcription at thermophilic temperatures, it is advantageous that this RT-PCR integrates RT step and PCR step into a single tube, and use Magnesium ion rather than Manganese ion for high fidelity of amplification.

RT-PAP: Thermostable purified DNA-dependent DNA polymerases also efficiently catalyze RNA-dependent DNA pyrophosphorolysis on an RNA template at thermophilic temperatures, virtually also being reverse transcriptases or RNA-dependent DNA polymerases. Besides reverse transcription at thermophilic temperatures, they are advantageous that RT-PAP integrates RT step and PAP step into a single tube, and use Magnesium ion for high fidelity of amplification.

### SUMMARY OF THE INVENTION

### Reverse Transcription

A thermophilic purified DNA-dependent DNA polymerase from Carboxydothermus pertinax was found to efficiently catalyze reverse transcription or RNA-dependent DNA polymerization on an RNA template, which is also a reverse transcriptase or an RNA-dependent DNA polymerase.

The reverse transcriptase comprises an amino acid sequence that has more than 85% sequence identity to DNA-dependent DNA polymerase of SEQ ID NO: 1.

The reverse transcriptase is selected from a group of reverse transcriptases consisting of SEQ ID NOs: 1-8.

The reverse transcriptase is a member of Type A family of DNA-dependent DNA polymerase.

The reverse transcriptase comprises a DNA polymerase A domain which amino acid sequence has more than 90% sequence identity to DNA polymerase A domain of SEQ ID NO: 1.

The DNA polymerase A domain is selected from a group of DNA polymerase A domains consisting of SEQ ID NOs: 1-8.

The reverse transcriptase activity is heat-inactivated at a temperature ≥83° C.

The reverse transcriptase activity is heat-inactivated at a temperature from 83° C. to 95° C.

The reverse transcriptase catalyzes reverse transcription at a temperature from 55° C. to 70° C.

The reverse transcriptase catalyzes reverse transcription at an optimal temperature from 60° C. to 62° C.

The reverse transcriptase catalyzes reverse transcription on an RNA template to generate a reverse-transcribed DNA, and then a second DNA polymerase catalyzes DNA-dependent DNA polymerization using the reverse-transcribed DNA as template.

A method for reverse transcription using RNA-dependent DNA polymerization comprising:
(a) providing a thermophilic purified DNA-dependent DNA polymerase from Carboxydothermus pertinax that catalyzes reverse transcription by RNA-dependent DNA polymerization on an RNA template, wherein the polymerase is also a reverse transcriptase or an RNA-dependent DNA polymerase comprising an amino acid sequence that has more than 85% sequence identity to DNA-dependent DNA polymerase of SEQ ID NO: 1, and
(b) catalyzing reverse transcription on an RNA template with the reverse transcriptase to generate a reverse-transcribed DNA, and
(c) the reverse transcriptase activity is heat-inactivated at a temperature ≥83° C.

Of the method for reverse transcription, the reverse transcriptase is selected from a group of reverse transcriptases consisting of SEQ ID NOs: 1-8.

Of the method for reverse transcription, the reverse transcriptase comprises a DNA polymerase A domain which amino acid sequence has more than 90% sequence identity to DNA polymerase A domain of SEQ ID NO: 1.

The DNA polymerase A domain is selected from a group of DNA polymerase A domains consisting of SEQ ID NOs: 1-8.

Of the method for reverse transcription, the reverse transcriptase activity is heat-inactivated at a temperature ≥83° C.

Of the method for reverse transcription, the reverse transcriptase activity is heat-inactivated at a temperature from 83° C. to 95° C.

Of the method, the reverse transcriptase catalyzes reverse transcription at a temperature from 55° C. to 70° C.

Of the method, the reverse transcriptase catalyzes reverse transcription at an optimal temperature from 60° C. to 62° C.

The method for reverse transcription further comprises another step (c) a second DNA polymerase catalyzing DNA-dependent DNA polymerization using the reverse-transcribed DNA as template.

Of the method for reverse transcription, the steps (a) to (c) are conducted in a single reaction tube.

### RNA-Dependent DNA Pyrophosphorolysis

A thermophilic purified DNA-dependent DNA polymerase from Carboxydothermus pertinax, that catalyzes RNA-dependent DNA pyrophosphorolysis on an RNA template, in which the polymerase is also a reverse transcriptase or an RNA-dependent DNA polymerase, comprises an amino acid sequence with more than 85% sequence identity to DNA-dependent DNA polymerase of SEQ ID NO: 1.

The reverse transcriptase contains a phenylalanine to tyrosine substation corresponding to position 665 of SEQ ID NO: 1.

The reverse transcriptase is selected from reverse transcriptases of SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8.

In addition, the reverse transcriptase comprises a DNA polymerase A domain which amino acid sequence has more than 90% sequence identity to DNA-dependent DNA polymerase of SEQ ID NO: 1.

The DNA polymerase A domain contains a substitution tyrosine corresponding to position 665 of SEQ ID NO: 1.

The DNA polymerase A domain is selected from DNA polymerase A domains of SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8.

When a 3′ blocked primer that has a non-extendable nucleotide at the 3′-blocker anneals to the RNA template, the reverse transcriptase catalyzes RNA-dependent DNA pyrophosphorolysis to remove the 3′ blocker from the 3′ blocked primer.

Then the reverse transcriptase catalyzes RNA-dependent DNA polymerization on the RNA template to extend the shortened 3′ blocked primer.

The reverse transcriptase activity is heat-inactivated at a temperature ≥83° C.

The reverse transcriptase activity is heat-inactivated at a temperature from 83° C. to 95° C.

The reverse transcriptase catalyzes RNA-dependent DNA pyrophosphorolysis at a temperature from 55° C. to 70° C.

The reverse transcriptase catalyzes RNA-dependent DNA pyrophosphorolysis at an optimal temperature from 60° C. to 62° C.

When a second 3′ blocked primer that has a non-extendable nucleotide at the 3′ blocker anneals to the reverse-transcribed DNA template, a second DNA-dependent DNA polymerase catalyzes DNA-dependent DNA pyrophosphorolysis to remove the 3′ blocker from the 3′ blocked primer and then catalyzes DNA-dependent DNA polymerization to extend the shortened primer.

A method for RNA-dependent DNA pyrophosphorolysis comprises:
(a) providing a thermophilic purified DNA-dependent DNA polymerase from Carboxydothermus pertinax that catalyzes RNA-dependent DNA pyrophosphorolysis on an RNA template, wherein the polymerase is also a reverse transcriptase or an RNA-dependent DNA polymerase comprising an amino acid sequence that has more than 85% sequence identity to DNA-dependent DNA polymerase of SEQ ID NO: 8;
(b) providing a 3′ blocked primer that has a non-extendable nucleotide at the 3′-blocker to anneal to an RNA template; and
(c) catalyzing RNA-dependent DNA pyrophosphorolysis with the reverse transcriptase to remove the 3′ blocker from the 3′ blocked primer.

Of the method for RNA-dependent DNA pyrophosphorolysis, the reverse transcriptase contains a tyrosine at position 420 of SEQ ID NO: 8.

Of the method, the reverse transcriptase is selected from reverse transcriptases of SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8.

Of the method, the reverse transcriptase comprises a DNA polymerase A domain which amino acid sequence has more than 90% sequence identity to DNA-dependent DNA polymerase of SEQ ID NO: 8.

Of the method, the DNA polymerase A domain is selected from DNA polymerase A domains of SEQ ID NO: 1, SEQ ID NO: 2, SEQ ID NO: 3, SEQ ID NO: 4, NO: 5, SEQ ID NO: 6, SEQ ID NO: 7 or SEQ ID NO: 8.

The method for RNA-dependent DNA pyrophosphorolysis further comprises step (d) catalyzing reverse transcription on the RNA template with the reverse transcriptase to generate a reverse-transcribed DNA.

Of the method, the reverse transcriptase activity is heat-inactivated at a temperature ≥83° C.

Of the method, the reverse transcriptase activity is heat-inactivated at a temperature from 83° C. to 95° C.

Of the method, the reverse transcriptase catalyzes RNA-dependent DNA pyrophosphorolysis at a temperature from 55° C. to 70° C.

Of the method, the reverse transcriptase catalyzes RNA-dependent DNA pyrophosphorolysis at an optimal temperature from 60° C. to 62° C.

Of the method for RNA-dependent DNA pyrophosphorolysis, the method further comprises steps:
(e) a second 3′ blocked primer that has a non-extendable nucleotide at the 3′ blocker anneals to the reverse-transcribed DNA template;
(f) a second DNA-dependent DNA polymerase that catalyzes DNA-dependent DNA pyrophosphorolysis to remove the 3′ blocker from the 3′ blocked primer to activate the primer; and
(g) a second DNA-dependent DNA polymerase that catalyzes DNA-dependent DNA polymerization using the reverse-transcribed DNA as template.

Of the method, the steps (a) to (g) are conducted in the same reaction tube.

### BRIEF DESCRIPTION OF THE DRAWINGS

FIG. 1 shows RT-PAP Assay V to detect COVID-19 viral RNA templates. To demonstrate the sensitivity, the amount of COVID-19 viral RNA was 10-fold serially diluted from 10,000 copies to a single digit of copies per reaction with 5 ng of reverse transcriptase Cpe-665Y used for reverse transcription step. X-axis is the cycle number and Y-axis is the net fluorescence signal in arbitrary units. Threshold line is also indicated. A linear relationship between the cope number of RNA template and its Ct value was set. In addition, non-template-controls showed no amplification signals, indicating the specificity.

FIG. 2 shows applications of RT-PAP Assays V and VI to clinical diagnostics. RT-PAP Assay V is used to amplify from a potential COVID-19 viral RNA template (Panels A, B and C). RT-PAP Assay VI is used to amplify from a potential control RNA template of the human ACTB gene (Panels D, E and F). Three COVID-19 positive RNA samples (S1 to S3), three COVID-19 negative RNA samples (S4 to S6), and three non-template controls were exampled. In addition, in melting curve analysis, the amplified products showed correct Tₘ values (Tm=81° C. with amplified COVID-19 products, and Tₘ=81° C. with amplified ACTB products).

### DETAILED DESCRIPTION OF THE INVENTION

### Terminology

Unless defined otherwise, all technical and scientific terms used herein have the same meaning as commonly understood by one of ordinary skill in the art.

Pyrophosphorolysis is the reverse reaction of deoxyribonucleic acid polymerization catalyzed by polymerase. In the presence of pyrophosphate, the 3′ nucleotide is removed by a polymerase on duplex DNA to generate a triphosphate nucleotide and a 3′ shortened duplex DNA (Deutscher and Kornberg, 1969):

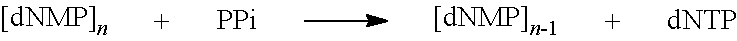

DNA-dependent DNA pyrophosphorolysis refers to pyrophosphorolysis of DNA using a DNA template. It is the reverse reaction of DNA-dependent DNA polymerization

RNA-dependent DNA pyrophosphorolysis refers to pyrophosphorolysis of DNA using an RNA template. It is the reverse reaction of RNA-dependent DNA polymerization.

3′ blocked primer refers to a primer with a 3′ non-extendable nucleotide (3′ blocker), such as a dideoxynucleotide or acyclonucleotide. The primer could not be directly extended from the 3′ blocker, but it can be removed by pyrophosphorolysis and then the unblocked primer can be extended by polymerase. Besides dideoxynucleotides, other 3′ blockers, such as acyclonucleotides that substitute a 2-hydroxyethoxymethyl group for the 2′-deoxyribofuranosyl sugar, are also used (Liu and Sommer, 2004a).

cDNA refers to a complementary DNA molecule synthesized on an RNA template.

Thermostable DNA polymerase that may be DNA-dependent or RNA-dependent refers to a polymerase that is heat stable or heat resistant at ≥50° C.

Thermophilic DNA polymerase that may be DNA-dependent or RNA-dependent refers to a polymerase with an optimal reaction temperature between 50° C. and 64° C.

Extreme thermophilic DNA polymerase that may be DNA-dependent or RNA-dependent refers to a polymerase with an optimal reaction temperature between 65° C. and 80° C.

Hyperthermophilic DNA polymerase that may be DNA-dependent or RNA-dependent refers to a polymerase with an optimal reaction temperature above 80° C.

DNA-dependent DNA polymerase refers to a polymerase characterized as catalyzing DNA polymerization or extension on a DNA template.

Reverse transcriptase or RNA-dependent DNA polymerase refers to a polymerase characterized as catalyzing DNA polymerization or extension on an RNA template.

Thermostable reverse transcriptase or RNA-dependent DNA polymerase refers to a reverse transcriptase that is heat stable or heat resistant at ≥50° C.

Thermophilic reverse transcriptase or RNA-dependent DNA polymerase refers to a reverse transcriptase with an optimal reaction temperature between 50° C. and 64° C.

Extreme thermophilic reverse transcriptase or RNA-dependent DNA polymerase refers to a reverse transcriptase with an optimal reaction temperature between 65° C. and 80° C.

Hyperthermophilic reverse transcriptase or RNA-dependent DNA polymerase refers to a reverse transcriptase with an optimal reaction temperature above 80° C.

Protein mutation refers to a change in amino acid residue at a location of a protein, like Cpe-665Y DNA polymerase. The change in amino acid residue is defined with respect to a naturally occurring protein such as Cpe DNA polymerase. A protein having a mutation is referred to as a “mutant” protein.

TaqFS is a mutant form of the wild-type Taq DNA polymerase containing F667Y amino acid changes compared with the wild-type DNA Taq sequence.

Cpe-665Y is a mutant form of Cpe DNA polymerase containing F665Y amino acid changes compared with the wild-type Cpe sequence.

A pair of primers means two opposing forward and reverse primers.

Singleplex-PAP means that one pair of primers amplify from one template in a reaction.

Multiplex-PAP means that >2 pairs of primers amplify from >2 potential templates in a reaction.

The 5′ region of a primer is the 5′ part of the primer sequence, such as the ten successive nucleotides from the 5′ end.

The 3′ region of a primer is the 3′ part of the primer sequence, such as the ten successive nucleotides from the 3′ end.

Central region of a primer is the middle part of the primer sequence between the 5′ region and the 3′ region.

Bidirectional-PAP (Bi-PAP) is a form of PAP that uses a pair of opposing 3′ blocked primers that overlap by one nucleotide at their 3′ termini.

Exponential-PAP is a form of PAP that uses a pair of two opposing forward and reverse primers for exponential product accumulation with cycles. At least one primer is a 3′ blocked primer.

Sensitivity or detection limit is defined as the smallest copy number of a template that generates a detectable product when the 3′ blocked primers match the template at the targeted nucleotide, such as the 3′ end.

Specificity is defined as the largest copy number of a template that generates an undetectable product when the 3′ blocked primers mismatch the template at the targeted nucleotide, such as the 3′ end.

Selectivity, the ratio of sensitivity to specificity, is defined as the ability to detect a small number of copies of the matched template in the presence of a large number of copies of mismatched templates without causing false positives.

### Terminology of Bioinformatics

Alignment is the process or result of matching up the amino acid residues of two or more protein sequences to achieve the maximal level of identity.

Substitution is a non-identical amino acid at a given position in an alignment.

Identity is the extent to which two (nucleotide or amino acid) sequences have the same residues at the same positions in an alignment, often expressed as a percentage.

Similarity is the extent to which nucleotide or protein sequences are related. Similarity between two sequences can be expressed as a percent of sequence identity and/or a percent of positive substitutions.

Gap is a space introduced into an alignment to compensate for insertions and deletions in one sequence relative to another.

### Terminology of Real-Time Fluorescence Detection

Baseline is the level of fluorescence signal during initial cycles. The low level can be considered as background or “noise” of the reaction.

Threshold is defined as the level of fluorescence signal that is significantly higher than baseline signal and can distinguish amplification signal from the background.

Ct (threshold cycle) is the cycle number at which the fluorescence signal crosses the threshold.

Amplification efficiency is defined as the percent of template that is amplified by the end of a cycle.

### Terminology of Reverse Transcription

Reverse transcription efficiency is defined as the percent of RNA template that is reverse-transcribed by the end of reaction.

Reverse transcriptase activity is defined as the ability to polymerize deoxynucleoside triphosphates using an RNA template within a given period of time.

Reverse transcriptase specific activity is defined as the ratio of the activity to the amount of reverse transcriptase.

### EXAMPLES

The practice of the present invention employs, unless otherwise indicated, common techniques of chemistry, molecular biology and recombinant DNA, e.g., those by Sambrook et al., Molecular Cloning, 2nd Ed. (Sambrook et al., 1989); Sambrook and Russell, Molecular Cloning, 3rd Ed. (Sambrook and Russell, 2001); Ausubel, Current Protocols in Molecular Biology (Ausubel, 1994).

### Example 1

### Purified Reverse Transcriptases

The present invention provides purified wild-type DNA-dependent DNA polymerases of Cpe, Cfe, Cis and Cpe-Del (SEQ ID NOS: 1-4) in full-length form or in N-terminal deletion form, which were sequenced previously in bacteria of Carboxydothermus pertinax (Accession: WP_075859958), Carboxydothermus ferrireducens (Accession: WP_034541897), Carboxydothermus islandicus (ACCESSION: WP_075865044) and Carboxydothermus pertinax (accession: WP_075859958), respectively.

The wild-type DNA-dependent DNA polymerases were identified as Type A DNA-dependent DNA polymerases by protein homology method with lengths of 831 AA (amino acid), 831 AA, 831 AA and 584 AA (SEQ ID NOS: 1-4), respectively. Among them, Cpe-Del (SEQ ID NO: 4) is a N-terminal deletion form in which the N-terminal 246 AA are deleted from Cpe (SEQ ID NO: 1).

The present invention also provides purified mutant DNA-dependent DNA polymerases of Cpe-665Y, Cfe-665Y, Cis-665Y and Cpe-Del-420Y (SEQ ID NOS: 5-8) in full-length form or in N-terminal deletion form, which contain F665Y (Phe665Try), F665Y, F665Y and F420Y substitutions corresponding to their wild-type counterpart sequences (SEQ ID NOS: 1-4). We designed this Phenylalanine to Tyrosine substitution and expected it to increase the efficiency of RNA-dependent DNA pyrophosphorolysis to remove a 3′ blocker from a 3′ blocked primer compared with their wild-type counterparts.

The mutant DNA-dependent DNA polymerases were also identified as Type A DNA-dependent DNA polymerases by protein homology method with lengths of 831 AA, 831 AA, 831 AA and 584 AA (SEQ ID NOS: 5-8), respectively. Among them, Cpe-Del-420Y (SEQ ID NO: 8) is a N-terminal deletion form in which the N-terminal 246 AA are deleted compared to Cpe-665Y with 831 AA length (SEQ ID NO: 5).

Besides DNA-dependent DNA polymerization, the purified wild-type and mutant DNA-dependent DNA polymerases were found to efficiently catalyze reverse transcription or RNA-dependent DNA polymerization using RNA templates at thermophilic temperatures, thus also being reverse transcriptases or RNA-dependent DNA polymerases in nature (EXAMPLES 3-6). These genes were cloned with the proteins expressed and purified for the purpose of reverse transcription for RT-PCR.

Furthermore, besides DNA-dependent DNA polymerization and reverse transcription, the purified wild-type and mutant DNA-dependent DNA polymerases (SEQ ID NOS: 1-8) were tested to catalyze RNA-dependent DNA pyrophosphorolysis, thus being reverse transcriptases or RNA-dependent DNA polymerases. The DNA polymerases, when they contain phenylalanine to tyrosine substitutions (SEQ ID NOS: 5-8), were found to increase substantially the efficiency of pyrophosphorolysis to remove a 3′ blocker, such as a dideoxynucleotide, from a 3′ blocked primer on an RNA template at thermophilic temperatures, (EXAMPLES 3-6). These genes were cloned with the proteins expressed and purified for the purpose of RT-PAP to catalyze RNA-dependent DNA pyrophosphorolysis.

### Comparison of Amino Acid Sequences Among the Reverse Transcriptases

Amino acid sequences of the above DNA polymerases (also being reverse transcriptases or RNA-dependent DNA polymerases) were aligned to explore conserved regions, which we assume to have functional importance. Specifically, BLAST (basic local alignment search tool) was used to compare each two amino acid sequences among the DNA polymerases.

In the alignment, the amino acid residues of each two DNA polymerase sequences were aligned to achieve the maximal level of identity. The identity, defined as the extent to which two DNA polymerase amino acid sequences have the same residues at the same positions, was searched and expressed as a percentage among each two of the wild-type DNA polymerases of Cpe, Cfe, Cis and Cpe-Del (SEQ ID NOS: 1-4) (Table 1) and among each two of the mutant DNA polymerases of Cpe, Cfe, Cis and Cpe-Del (SEQ ID NOS: 5-8) (Table 2). For example, 831 amino acids were aligned between Cpe and Cfe DNA polymerase sequences, and 723 of the 831 amino acids, or 87.00%, were found identical at the same positions. The minimum identity was aligned to be 85.10%, which is between Cpe-del and Cis polymerase sequences among the wild-type DNA polymerases (Table 1) or between Cpe-del-420Y and Cis665Y polymerase sequences among the mutant DNA polymerases (Table 2), suggesting that as low as 85.10% identity or conservation level does not affect the DNA polymerase (also being reverse transcriptases or RNA-dependent DNA polymerases) functions substantially.

The gap, defined as one or more amino acid residues deleted or inserted between each two polymerase amino acid sequences was also searched and expressed as a percentage among each two of the wild-type DNA polymerases of Cpe, Cfe, Cis and Cpe-Del (SEQ ID NOS: 1-4) and among each two of the mutant DNA polymerases of Cpe-665Y, Cfe-665Y, Cis-665Y and Cpe-Del-420Y (SEQ ID NOS: 5-8).

No gaps (0%) were found between each two DNA polymerase sequences among the wild-type polymerases or among the mutant DNA polymerases. For example, between Cpe and Cfe DNA polymerase sequences, 831 amino acids were aligned, and zero of the 831 amino acid residues, or 0%, were found deleted or inserted, suggesting that a gap is likely to affect the DNA polymerase (also being reverse transcriptases or RNA-dependent DNA polymerases) functions substantially.

To confirm the above findings of alignments, experiments were conducted in EXAMPLES 3-6. Although as low as 85% identity among their amino acid sequences (Tables 1 and 2), the DNA polymerases showed the activities to catalyze reverse transcription or RNA-dependent DNA polymerization and RNA-dependent DNA pyrophosphorolysis

Furthermore, although the N-terminal 246 AA was deleted from its full-length form, the DNA polymerase of Cpe-Del or Cpe-Del-420Y still kept the activities for reverse transcription and RNA-dependent DNA pyrophosphorolysis, showing that the N-terminal regions are not essential for the activities (EXAMPLES 3-6).

TABLE 1
Amino acid identities among the wild-type DNA polymerases

| | | SEQ ID | | | |
|---|---|---|---|---|---|
| | | NO: 1 | NO: 2 | NO: 3 | NO: 4 |
| | Polymerase | Cpe | Cfe | Cis | Cpe-Del |
| NO: 1 | Cpe | | | | |
| NO: 2 | Cfe | 87.00%, | | | |
| | | 723/831 ^{a} | | | |
| NO: 3 | Cis | 85.80%, | 94.83%, | | |
| | | 713/831 | 788/831 | | |
| NO: 4 | Cpe-del | 100%, | 86.82%, | 85.10%, | |
| | | 584/584 | 507/584 | 497/584 | |

Footnotes of Table 1.
^{a} 831 amino acids were aligned between Cpe and Cfe DNA polymerase sequences. 723 of the 831 amino acids, or 87.00%, were found identical at the same positions.

TABLE 2
Amino acid identities among the mutant DNA polymerases

| | | SEQ ID | | | |
|---|---|---|---|---|---|
| | | | | NO: 7 | NO: 8 |
| | | NO: 5 | NO: 6 | Cis- | Cpe-Del- |
| SEQ ID | Polymerase | Cpe-665Y | Cfe-665Y | 665Y | 420Y |
| NO: 5 | Cpe-665Y | | | | |
| NO: 6 | Cfe-665Y | 87.00%, | | | |
| | | 723/831 | | | |
| NO: 7 | Cis-665Y | 85.80%, | 94.83%, | | |
| | | 713/831 | 788/831 | | |
| NO: 8 | Cpe-Del- | 100%, | 86.82%, | 85.10%, | |
| | 420Y | 584/584 | 507/584 | 497/584 | |

On the other hand, we identified functional domains of each DNA polymerase (also being reverse transcriptase or RNA-dependent DNA polymerase) including 1) a 5′-3′ exonuclease domain (a total of 259 amino acids from the 2nd to the 260th amino acid of the 831 amino acid sequence), and 2) a DNA polymerase A domain which sustains the activity for polymerization (a total of 373 amino acids from the 452nd to the 824th amino acid of the 831 amino acid sequence) by protein homology method with E. coli DNA polymerase A.

The identity of the DNA polymerase A domains was expressed as a percentage among each two of the wild-type DNA polymerases of Cpe, Cfe, Cis and Cpe-Del (SEQ ID NOS: 1-4) (Table 3) and among each two of the mutant DNA polymerases of Cpe, Cfe, Cis and Cpe-Del (SEQ ID NOS: 5-8) (Table 4).

For example, the 373 amino acids of the DNA polymerase A domains were aligned between Cpe and Cfe DNA polymerase sequences, and 341 of the 373 amino acids, or 91.42%, were found identical at the same positions. The minimum identity was aligned to be 90.35%, which is between Cpe or Cpe-del and Cis DNA polymerase sequences among the wild-type DNA polymerases (Table 3) or between Cpe-665Y or Cpe-del-420Y and Cis665Y polymerase sequences among the mutant DNA polymerases (Table 4), suggesting that as low as 90.35% identity or conservation level does not affect the DNA polymerase activities substantially.

The gap between each two amino acid sequences of the DNA polymerase A domains was also expressed as a percentage among each two of the wild-type DNA polymerases of Cpe, Cfe, Cis and Cpe-Del (SEQ ID NOS: 1-4) and among each two of the mutant DNA polymerases of Cpe-665Y, Cfe-665Y, Cis-665Y and Cpe-Del-420Y (SEQ ID NOS: 5-8).

No gaps (0%) were found between each two DNA polymerase A domains. For example, between Cpe and Cfe DNA polymerase sequences, 373 amino acids were aligned, and zero of the 373 amino acid residues, or 0%, were found deleted or inserted, suggesting that a gap is likely to affect the DNA polymerase activities substantially.

Besides the alignments of DNA polymerase A domains, the importance of these domains was also demonstrated in EXAMPLES 3-6. Although as low as 90% identity among their DNA polymerase A domains (Tables 3 and 4), the DNA polymerases showed RNA-dependent DNA polymerase or reverse transcriptase activities to catalyze reverse transcription or RNA-dependent DNA polymerization and RNA-dependent DNA pyrophosphorolysis.

Furthermore, after Cpe-Del (SEQ ID NO: 4) and Cpe-Del-420Y (SEQ ID NO: 8) have deleted their 5′-3′ exonuclease domains, they still sustained their activities of reverse transcription and RNA-dependent DNA pyrophosphorolysis, showing again that their 5′-3′ exonuclease domains play unessential roles and their DNA polymerase A domains play essential roles in their activities for reverse transcription and RNA-dependent pyrophosphorolysis (EXAMPLES 3-6).

TABLE 3
Amino acid identities among the DNA polymerase A
domains of the wild-type DNA polymerases

| | | SEQ ID | | | |
|---|---|---|---|---|---|
| | | NO: 1 | NO: 2 | NO: 3 | NO: 4 |
| | Polymerase | Cpe | Cfe | Cis | Cpe-Del |
| NO: 1 | Cpe | | | | |
| NO: 2 | Cfe | 91.42%, | | | |
| | | 341/373 ^{a} | | | |
| NO: 3 | Cis | 90.35%, | 96.51%, | | |
| | | 337/373 | 360/373 | | |
| NO: 4 | Cpe-del | 100%, | 91.42%, | 90.35%, | |
| | | 373/373 | 341/373 | 337/373 | |

Footnotes of Table 3.
^{a} 373 amino acids were aligned between Cpe and Cfe DNA polymerase sequences. 341 of the 373 amino acids, or 91.42%, were found identical at the same positions.

TABLE 4
Amino acid identities among the DNA polymerase A
domains of the mutant DNA polymerases

| | | SEQ ID | | | |
|---|---|---|---|---|---|
| | | | | NO: 7 | NO: 8 |
| | | NO: 5 | NO: 6 | Cis- | Cpe-Del- |
| SEQ ID | Polymerase | Cpe-665Y | Cfe-665Y | 665Y | 420Y |
| NO: 5 | Cpe-665Y | | | | |
| NO: 6 | Cfe-665Y | 91.42%, | | | |
| | | 341/373 | | | |
| NO: 7 | Cis-665Y | 90.35%, | 96.51%, | | |
| | | 337/373 | 360/373 | | |
| NO: 8 | Cpe-Del- | 100%, | 91.42%, | 90.35% | |
| | 420Y | 373/373 | 341/373 | 337/373 | |

### Example 2

### Construction of Recombinant Plasmids

The present invention provides nucleic acid sequences encoding the wild-type DNA polymerases (reverse transcriptases) (SEQ ID NOS: 9-12). They are also available from GenBank of Cpe: Accession: NZ_BDJK01000055, Region: 138926 . . . 141421; Cfe: Accession: NZ_ATYG01000019, Region: 19948 . . . 22443; Cis: Accession: NZ_BDJL01000019, Region: 39893 . . . 42388; Cpe-Del: Accession: NZ_BDJK01000055, Region: 139664 . . . 141421. It also provides nucleic acid sequences encoding the mutant reverse transcriptases (SEQ ID NOS: 13-16), in each of which an amino acid is substituted to contain F665Y, F665Y, F665Y and F420Y, respectively.

A wild-type or mutant DNA polymerase gene of a pair was chosen (SEQ ID NOS: 1-8). The nucleic acid codons of the gene were optimized for a high level of expression in E. coli cells, and then its DNA sequence was chemically synthesized with adapters at each end (GenScript Biotech). To construct a recombinant plasmid, the DNA sequence was then ligated into pET vector DNA at the Nde I and BamH I restriction sites according to Navagen pET system user manual. For convenient purification, the expressed polymerase contained 6× or 10× His-Tag at the N-terminus or C-terminus (Table 5).

The counterpart recombinant plasmid DNA of the pair was constructed by site-directed mutagenesis method with QuikChange lightning site-directed mutagenesis kit according to Agilent's user manual so that a substitution change was introduced into the polymerase gene (Table 5).

Then each recombinant plasmid DNA transformed DH5a. E. coli cells and its plasmid DNA was extracted from E. coli colonies. The DNA sequence of the gene was sequenced by ABI Sanger sequencing.

### Expression of the Reverse Transcriptases

Each cloned polymerase was expressed in transformed T7 Express lysY/I^{q} E. coli cells according to BioLabs' manual, including steps: 1) transformation of E. coli cells, 2) a small volume of LB culture of E. coli colonies, 3) a large volume of LB culture of the E. coli cells, 4) IPTG induction of protein expression in the E. coli cells, and 5) collection of the cultured E. coli cells by centrifuge

TABLE 5
Characteristics of the constructed recombinant plasmids

| Inserted | | Bacteria and | Tag for |
|---|---|---|---|
| polymerase gene | Vector | expression | purification |
| Cpe, Cpe-665Y: | pET-16b, between | E.coli, | 10X Histidines |
| SEQ ID NOS: 1, 5 | Nde I and BamH I | T7 Express | at N-terminal |
| | restriction sites | system | region |
| Cfe, Cfe-665Y: | pET-16b, between | E.coli, | 10X Histidines |
| SEQ ID NOS: 2, 6 | Nde I and BamH I | T7 Express | at N-terminal |
| | restriction sites | system | region |
| Cis, Cis-665Y: | pET-20b, between | E.coli, | 6X Histidines |
| SEQ ID NOS: 3, 7 | Nde I and BamH I | T7 Express | at C-terminal |
| | restriction sites | system | region |
| Cpe-Del, Cpe- | pET-14b, between | E.coli, | 6X Histidines |
| Del-665Y ^{a}: SEQ | Nde I and BamH I | T7 Express | at N-terminal |
| ID NOS: 4, 8 | restriction sites | system | region |

Footnotes of Table 5.
^{a} Six nucleotides of 5′ ATGGGC3′ coding Methionine and Glycine amino acids at the N-terminus was added to the 5′ end of the gene sequence for convenient expression.

### Purification of the Reverse Transcriptases

Because an expressed polymerase contained 6× or 10× His-Tags at the N-terminus or C-terminus, His-Tag affinity chromatography was chosen for the DNA polymerase purification. A HisTrap HP column was used under native conditions with an AKTA RFLP instrument according to manufacturer's instruction (GE lifesciences).

Within the HisTrap HP column, the chelating group, attached to agarose beads, is pre-charged with nickel ion, which selectively binds the histidine-Tag polymerase. Then 250 mM Imidazole elutes the bound polymerase through competitive binding.

The purification procedure included four basic steps: 1) cell lysis, 2) polymerase binding to the HisTrap HP column, 3) washing and 4) step-elution. Two buffers A and B were used: A buffer contained 50 mM KH₂PO₄/K₂HPO₄, pH 8.0, 300 mM NaCl, 10 mM Imidazole used for steps 1) to 3), and B buffer contained 50 mM KH₂PO₄/K₂HPO₄, pH 8.0, 300 mM NaCl, 250 mM Imidazole used for step 4). Then the eluted polymerase was concentrated by centrifuging through a Millipore Amicon Ultra 10K ultra-filtration column.

A typical yield of the eluted polymerase was 4-20 mg from 1000 ml of E. coli culture. The purified polymerase showed one major band on SDS-PAGE gel after Coomassie Blue staining, indicating ≥90% purity. Based on the gel mobility, the major band was estimated 95,000 Daltons for a full-length polymerase and 67,000 Daltons for a deleted polymerase.

The purified polymerase was stored at 1 mg per mL of storage buffer, containing 20 mM Tris-HCl (pH 8.0 at 25° C.), 100 mM KCl, 0.1 mM EDTA, 1 mM DTT, 0.2% Tween-20 and 50% Glycerol at −20° C.

### Example 3

### Preparation of Primers

Each 3′ blocked primer, blocked with ddCMP at the 3′ end, was chemically synthesized and HPLC purified by Integrated DNA Technologies. The regular DNA primers were also synthesized by the same vendor (Table 6).

TABLE 6
List of primers and assays

| | | Sequence (5′ to 3′) | | Product | Starting |
|---|---|---|---|---|---|
| Assay | Name ^{b} | (SEQ ID NO:) | Location | size (bp) | template |
| I. RT- | ACTB(426)25D | CAACCGCGAGAAGATGA | Exons | 63 | Total |
| PCR ^{a} | | CCCAGATC (17) | 3 and 4 | | RNA |
| | ACTB(488)25U | GCAACGTACATGGCTGG | Exon 4 | | |
| | | GGTGTTGA (18) | | | |
| II. | ACTB(426)25D | CAACCGCGAGAAGATGA | Exons | 280 | Total |
| RT- | | CCCAGATC (19) | 3 and 4 | | RNA |
| PCR | ACTB(705)25U | TTCCCGCTCGGCCGTGGT | Exon 4 | | |
| | | GGTGAAG (20) | | | |
| III. | ACTB(426)25D | CAACCGCGAGAAGATGA | Exons | 63 | Total |
| RT- | | CCCAGATC (21) | 3 and 4 | | RNA |
| PAP | ACTB(488)30U | GCAACGTACATGGCTGG | Exon 4 | | |
| | | GGTGTTGAAGGTddC (22) ^{c} | | | |
| IV. | ACTB(426)25D | CAACCGCGAGAAGATGA | Exons | 147 | Total |
| RT- | | CCCAGATC (23) | 3 and 4 | | RNA |
| PAP | ACTB(572)30U | ACAGTGTGGGTGACCCC | Exon 4 | | |
| | | GTCACCGGAGTCddC (24) | | | |
| V. | COVID- | GAGGGAGCCTTGAATAC | N | 76 | Clinical |
| RT- | 19(28679)28D | ACCAAAAGATddC (25) | region | | RNA |
| PAP | COVID- | GTTGTAGCACGATTGCAG | N | | sample |
| | 19(28754)28U | CATTGTTAGddC (26) | region | | |
| VI. | ACTB(434)28D | AGAAGATGACCCAGATC | Exons | 80 | Clinical |
| RT- | | ATGTTTGAGAddC (27) | 3 and 4 | | RNA |
| PAP | ACTB(512)27U | TTACAGGGATAGCACAG | Exon 4 | | sample |
| | | CCTGGATAGddC (28) | | | |

Footnoots of Table 6.
^{a} In order to avoid non-specific amplification from potentially contaminated genomic DNA, RT-PCR and RT-PAP primers were designed to anneal to regions that span introns of the human ACTB gene.
^{b} For example, ACTB means the human ACTB gene; (426), 5′ end of the primer begins at nucleotide 426; D, downstream (i.e., in the direction of transcription). The ACTB mRNA is from GenBank accession: NM_001101.3. The COVID-19 viral RNA is from GenBank accession: MN908947.
^{c} Due to the availability of chemical synthesis, ddCMP was exampled.

### Total RNA Extraction

Total RNA was extracted from human blood white cells using QIAamp RNA kit according to Qiagen′ protocol (QIAamp RNA Blood Mini Handbook). Within the process, RNase-Free DNase was used to remove contaminated genomic DNA. The concentration of the total RNA was measured by a spectrophotometer at 260 nm. It was stored at −20° C. until use.

### Clinical RNA Sample Extraction

Clinical COVID-19 positive and negative samples were collected from nasopharyngeal or oropharyngeal swabs from the upper respiratory system. The RNA of a sample was extracted using QIAamp Viral RNA Mini Kits (Qiagen) following the manufacturer's instructions. The starting volume of the sample was 200 μL and the elution volume was 50 μL. Besides the COVID-19 viral RNA, the eluted sample contained co-extracted human RNA. The quantity of the extracted sample was measured by a EUA approved COVID-19 viral RNA diagnostic RT-PCR kit. It was stored at −20° C. until use.

### RT-PCR to Measure Reverse Transcriptase Activity

Unless stated otherwise, the RT-PCR reaction mixture of 20 μl contained 50 mM Tris-HCl (pH 8.8 at 25° C.), 10 mM (NH₄)₂SO₄, 1.5 mM MgCl₂, 200 μM each dNTPs (dATP, dTTP, dGTP and dCTP), 0.1-0.15 μM each primer of RT-PCR Assay I or II, 0.1×SYBR Green I dye, 0.02% Twee-20, 5-200 ng of the reverse transcriptase, 1.6 U Taq DNA polymerase and 0.2 ng total RNA template. For RT-PCR, the RT step (reverse transcription) and the PCR step (DNA-dependent DNA polymerization) were integrated into a single reaction tube.

A Bio-Rad CFX96 real time PCR detection system was used for quantification of the amplified product. Analysis mode: SYBR Green fluorophore, Baseline setting: baseline subtracted curve fit, Threshold cycle (Ct) determination: single threshold, Baseline method: SYBR auto calculated, Threshold setting: auto calculated.

For RT-PCR assays I and II, the program entailed 60° C. for 10 minutes for reverse transcription; then 95° C. for 2 minutes for heat-inactivation of reverse transcriptase activity; and then cycling 95° C. for 10 seconds, 60° C. for 30 seconds, 72° C. for 1 minute for 35 cycles. To confirm the amplified product, melting curve analysis was followed from 72° C. to 95° C. with increment 1° C. and holding 5 seconds.

### RT-PAP to Measure Reverse Transcriptase Activity

Unless stated otherwise, the PAP reaction mixture of 20 μl contained 50 mM Tris-HCl (pH 8.0 at 25° C.), 10 mM (NH₄)₂SO₄, 1.0-1.2 mM MgCl₂, 25 μM each dNTPs (dATP, dTTP, dGTP and dCTP), 0.10-0.15 μM each primer of RT-PAP Assay III, IV, V or VI, 90 M Na₄PPᵢ, 0.1×SYBR Green I dye, 0.02% Twee-20, 5-200 ng the reverse transcriptase, 1.6 U TaqFS DNA polymerase and 0.2 ng total RNA template or 5 ul COVID-19 viral RNA sample. For RT-PAP, the RT step (RNA-dependent DNA pyrophosphorolysis and reverse transcription) and the PAP step (DNA-dependent DNA pyrophosphorolysis and DNA-dependent DNA polymerization) were integrated into a single reaction tube.

A Bio-Rad CFX96 real time PCR detection system was used for quantification of the amplified product. Analysis mode: SYBR Green fluorophore, Baseline setting: baseline subtracted curve fit, Threshold cycle (Ct) determination: single threshold, Baseline method: SYBR auto calculated, Threshold setting: auto calculated.

For RT-PAP Assays III and IV, the program entailed 60° C. for 10 minutes for reverse transcription; then 95° C. for 2 minutes for heat-inactivation of reverse transcriptase activity; and then cycling 95° C. for 10 seconds, 60° C. for 30 seconds, 72° C. for 1 minute for 35 cycles. To confirm the amplified product, melting curve analysis was followed from 72° C. to 95° C. with increment 1° C. and holding 5 seconds.

For RT-PAP Assays V and IV, the program entailed 60° C. for 10 minutes for reverse transcription; 95° C. for 2 minutes for heat-inactivation of reverse transcriptase activity; and then cycling 95° C. for 10 seconds, 64° C. for 1 minute for 45 cycles. To confirm the amplified product, melting curve analysis was followed from 64° C. to 95° C. with increment 1° C. and holding 5 seconds to confirm the amplified product.

### Example 4

### RT-PCR

RT-PCR includes two steps: 1) reverse transcription of an RNA template to produce a cDNA catalyzed by a reverse transcriptase, and 2) PCR amplification of the cDNA as template catalyzed by a DNA-dependent DNA polymerase, preferably in the same single reaction tube. Reverse transcription is considered as the efficiency limit of the two-step process.

The wild-type and mutant DNA polymerases (also being reverse transcriptases or RNA-dependent DNA polymerases) of Cpe, Cfe, Cis and Cpe-Del (SEQ ID NOS: 1-4) as well as Cpe-665Y, Cfe-665Y, Cis-665Y and Cpe-Del-420Y (SEQ ID NOS: 5-8) were tested for reverse transcription in RT-PCR.

We designed two RT-PCR Assays I and II (Table 6). RT-PCR Assay I amplifies from a 63-nt ACTB mRNA template with primers ACTB(426)25D and ACTB(488)25U (SEQ ID NOS: 17 and 18), and RT-PCR Assay II amplifies from a 280-nt ACTB mRNA template with primers ACTB(426)25D and ACTB(705)25U (SEQ ID NOS: 19 and 20). Each assay has a forward primer and a reverse primer with the reverse primer matching the RNA template. Unless stated otherwise, the reaction composition and thermocycling program are described in EXAMPLE 3.

### Heat-Inactivation

Initially we found that when a denaturing step at 94° C. or 95° C. for 2 minutes was added as reverse transcription step, any wild-type or mutant reverse transcriptase could not amplify from RNA templates, demonstrating that the reverse transcriptase could not tolerate 94° C. or a higher temperature even for a short period of time.

To test the heat-inactivation in detail, we examined the effect of various temperatures on the reverse transcriptases. In a special program, gradient temperatures from 83° C. to 94° C. for 2 minutes were added prior to reverse transcription step.

With RT-PCR Assay I, after heated at 83° C. for 2 minutes, a very weak amplification signal (+), indicated by a much delayed Ct value and a much lower RFU (relative fluorescent unit), was observed, estimating that >90% of reverse transcriptase activity was heat-inactivated (Left part of Table 7). With RT-PCR Assay II, no amplification signal was found after heated at 83° C. for 2 minutes. This difference is because the product size of RT-PCR Assay II is 280 nt, much larger than 63 nt of RT-PCR Assay I. When at 85.3° C. or higher temperatures, no amplification signals were observed with both RT-PCR Assays I and II, estimating that >99% of reverse transcriptase activities were heat-inactivated (Left part of Table 7).

The reverse transcriptases showed similar properties of heat-inactivation, although as low as 85% identity among their amino acid sequences (Tables 1 and 2) and as low as 90% identity among their DNA polymerase A domains (Tables 3 and 4).

In addition, non-template-control or non-reverse-transcriptase-control did not show amplifications, indicating the specificity.

This heat-inactivation is useful to RT-PCR because it prevents the RT step from interacting with the PCR step when they are integrated into a single tube.

TABLE 7
Remaining reverse transcriptase activities after heat-activation

| | Amplification signal and estimated remaining activity | | | |
|---|---|---|---|---|
| | | RT-PCR | | RT-PAP |
| Heat | RT-PCR assay | assay | RT-PAP assay | assay |
| temperature | I ^{a} | II ^{a} | III ^{c} | IV ^{c} |
| 94.0° C. | −, NA ^{b} | −, NA | −, NA ^{a} | −, NA |
| 90.0° C. | −, NA | −, NA | −, NA | −, NA |
| 87.4° C. | −, NA | −, NA | −, NA | −, NA |
| 85.3° C. | −, NA | −, NA | −, NA | −, NA |
| 83.0° C. | +/−, 5-10% | −, NA | +/−, 5-10% | −, NA |

Footnotes of Table 7
^{a} In the left part, with RT-PCR assays I and II, 5 ng and 10 ng of Cpe polymerase was added to a 20 ul of reaction, respectively.
^{b} Amplification signals can be divided into five levels of +++, ++, +, +/− and −. +/− means very weak amplification and − means no amplification initiated by the reverse transcriptases. The remaining activity was estimated from the amplification signal, indicated by its Cₜ value and RFU (relative fluorescent unit). NA means not available due to too low level of the remaining activity, such as <1%.
^{c} In the right part, with RT-PAP assays III and IV, 5 ng and 10 ng of Cpe-665Y polymerase was added to a 20 ul of reaction, respectively (EXAMPLE V).

### Optimal Temperature

To test the optimal temperature, we examined the effect of various reaction temperatures from 50° C. to 75° C. A special program entailed gradient temperatures from 56° C. to 66.5° C. for 10 minutes for reverse transcription step.

RT-PCR Assay II is a larger and more difficult target for reverse transcription, and thus it reflects the optimal temperature more accurately than RT-PCR Assay I.

With RT-PCR Assay II, each reverse transcriptase was tested to measure Ct values at different temperatures (Left part of Table 8). Because Cₜ values are proportional to the reverse transcriptase activities or efficiencies, the Cₜ values can represent the reverse transcriptase activities or efficiencies. For comparison of the Cₜ values at the different temperatures, a ΔCₜ is set which equals to a Cₜ value subtracts that at 60.1° C. (Left part of Table 8). The results showed that the reverse transcriptases catalyzed reverse transcription in the RT-PCR assays substantially from 56° C. to 66.5° C. (Left part of Table 8). At 60.1-62.7° C., the Cₜ values were earliest and the ΔCₜ values were the smallest, indicating the reverse transcription activities or efficiencies to be highest at 60.1-62.7° C.

In addition, other tests showed that the reverse transcriptases still had observable activities at 55° C. and at 70° C.

Furthermore, non-template-control or non-reverse-transcriptase-control did not show amplifications, indicating the specificity.

The reverse transcriptases showed similar results of optimal temperatures, although as low as 85% identity among their amino acid sequences (Tables 1 and 2) and as low as 90% identity among their DNA polymerase A domains (Tables 3 and 4).

TABLE 8
Optimal temperatures of reverse transcription

| RT reaction | RT-PCR assay II | | RT-PAP assay IV | |
|---|---|---|---|---|
| temperature | Cₜ ^{a} | ΔCt to 60.1° C. ^{b} | Cₜ ^{c} | ΔCₜ to 60.1° C. |
| 66.5° C. | 25.6 | 3.5 | 22.6 | 2.3 |
| 65.9° C. | 25.0 | 2.9 | 22.0 | 1.7 |
| 64.8° C. | 23.3 | 1.2 | 21.1 | 0.8 |
| 62.7° C. | 22.2 | 0.1 | 20.2 | −0.1 |
| 60.1° C. | 22.1 | 0 | 20.3 | 0 |
| 58.1° C. | 23.0 | 0.9 | 21.4 | 1.1 |
| 56.7° C. | 24.2 | 2.1 | 22.4 | 2.1 |
| 56.0° C. | 25.8 | 3.7 | 23.2 | 2.9 |

Footnotes of Table 8.
^{a} In the left part with RT-PCR assay II, a Cₜ value was obtained when 10 ng of Cpe DNA polymerase was added to a 20 ul of reaction.
^{b} For comparison of the Cₜ values at the various temperatures more conveniently, a ΔCt is set which equals to a Cₜ value subtracts that at 60.1° C.
^{c} In the left part with RT-PAP assay IV, a Cₜ value was obtained when 5 ng of Cpe-665Y DNA polymerase was added to a 20 ul of reaction (EXAMPLE V).

### Specific Activity

In order to compare relative activities for reverse transcription among the reverse transcriptases, a term of specific activity is introduced which is defined as the minimum amount (ng) of a reverse transcriptase that has an almost 100% efficiency in reverse transcription step.

RT-PCR Assay II was chosen because it is a larger and more difficult target and can measure the level of reverse transcriptase activity more obviously than RT-PCR Assay I.

We tested and compared the specific activities of the reverse transcriptases. The amount of each reverse transcriptase was 2-fold serially diluted from up to 200 ng to 1 ng in 20 μl of reaction. The Cₜ value was determined to estimate the minimum amount of the reverse transcriptase which has an almost 100% efficiency in the reverse transcription step. For comparison of the relative activities among the reverse transcriptases, the ratio of the minimum amount of a reverse transcriptase to that of Cpe was calculated (Left part of Table 9). The reverse transcriptases showed various specific activities with Cpe DNA polymerase having the highest specific activity among them.

In melting curve analysis, only one melting peak showed the Tm value to be 88° C. of the amplified product in RT-PAR Assay II. Non-template-control or non-reverse-transcriptase-control did not show any measurable Cₜ, showing specificity of the amplification.

In addition, when RT-PCR Assay I was used, the specific activities were higher with less amounts of the reverse transcriptases required than those in RT-PCR Assay II.

The reverse transcriptases showed various specific activities with the particular reverse transcriptases and with the particular RT-PCR assays.

TABLE 9
Specific activities of the reverse transcriptases

| RT-PCR Assay II | | | RT-PAP Assay IV | | |
|---|---|---|---|---|---|
| | The | | | The | Ratio to |
| | minimum | Ratio | | minimum | Cpe- |
| Polymerase | amount ^{a} | to Cpe ^{b} | Polymerase | amount ^{a} | 665Y ^{b} |
| Cpe | 10 ng | 1 | Cpe-665Y | 5 ng | 1 |
| Cfe | 20 ng | 2 | Cfe-665Y | 10 ng | 2 |
| Cis | 80 ng | 8 | Cis-665Y | 40 ng | 8 |
| Cpe-Del | 60 ng | 6 | Cpe-Del-665Y | 20 ng | 4 |

Footnotes of Table 9.
^{a} The amount of a reverse transcriptase was 2-fold serially diluted from 160 ng to 1.25 ng in 20 μl of reaction and incubated at 60° C. for 10 minutes in reverse transcription step. The Cₜ value was determined with the minimum amount of the reverse transcriptase estimated. The minimum amount of a reverse transcriptase is defined to have a Cₜ value of an almost 100% efficiency of reverse transcription.
^{b} For comparison of the specificity activities among the reverse transcriptases, the ratio of the minimum amount of a reverse transcriptase to that of Cpe or Cpe-665Y is set.

### Example 5

### RT-PAP

RT-PAP includes two steps of RT and PAP. 1) Using an RNA template, a reverse transcriptase or RNA-dependent DNA polymerase removes a 3′ blocker from a first 3′ blocked primer (RNA-dependent DNA pyrophosphorolysis), and then extends the unblocked primer to generate a DNA product (RNA-dependent DNA polymerization). 2) Using the DNA product as template, a DNA-dependent DNA polymerase removes another 3′ blocker from a second 3′ blocked primer (DNA-dependent DNA pyrophosphorolysis), and then extends the unblocked primer (DNA-dependent DNA polymerization). The first step, including RNA-dependent DNA pyrophosphorolysis and reverse transcription, is considered as the efficiency limit of the two-step process.

The wild-type and mutant DNA polymerases (also being reverse transcriptases or RNA-dependent DNA polymerases) (SEQ ID NOS: 1-8) were tested to catalyze RNA-dependent DNA pyrophosphorolysis and reverse transcription in RT-PAP. The DNA polymerases, when they contain phenylalanine to tyrosine substitutions (SEQ ID NOS: 5-8), were found to increase substantially the efficiencies of RNA-dependent DNA pyrophosphorolysis to remove the 3′ blockers, such as a dideoxynucleotide, from the 3′ blocked primer on an RNA template.

We designed four RT-PAP Assays III, IV, V and VI (Table 6) to amplify from RNA templates. Each assay had a forward primer and a reverse primer for exponential amplification. RT-PAP Assay III amplifies from a 63-nt ACTB mRNA template with primers ACTB(426)25D and ACTB(488)30U (3′ blocked primer) (SEQ IN NO: 21 and 22). RT-PAP Assay IV amplifies from a 147-nt ACTB mRNA template with primers ACTB(426)25D and ACTB(572)3OU (3′ blocked primer) (SEQ IN NO: 23 and 24). RT-PAP Assay V amplifies from a 76 nt COVID-19 viral RNA template with 3′ blocked primers COVID-19(28679)28D and COVID-19(28754)28U (SEQ IN NO: 25 and 26). RT-PAP Assay VI amplifies from a 80 nt ACTB mRNA template with 3′ blocked primers ACTB(434)28D and ACTB(512)27U (SEQ IN NO: 27 and 28). The reverse primers, blocked by dideoxynucleotides at the 3′ ends, matched the RNA templates.

Unless stated otherwise, the reaction composition and thermocycling program are described in EXAMPLE 3.

### Heat-Inactivation

To test the heat-inactivation in detail, we examined the effect of various temperatures on the mutant reverse transcriptases. A special program included gradient temperatures from 83° C. to 94° C. prior to reverse transcription step.

With RT-PAP Assay III, after heated at 83° C. for 2 minutes, a very weak amplification signal (±), indicated by a much delayed Cₜ value and a much lower RFU (relative fluorescent unit), was observed, estimating that >90% of reverse transcriptase activity was heat-inactivated (Right part of Table 7). With RT-PAP Assay IV, after heated at 83° C. for 2 minutes, no amplification signal was found. The difference is caused by their product sizes. With RT-PAP Assays III and IV, at 85.3° C. or higher temperatures, no amplification signals were observed, estimating that >99% of reverse transcriptase activities were heat-inactivated (Right part of Table 7).

The reverse transcriptases showed similar levels of remaining reverse transcriptase activities although as low as 85% identity among their amino acid sequences (Tables 1 and 2) and as low as 90% identity among their DNA polymerase A domains (Tables 3 and 4).

### Optimal Temperature

To test the optimal temperature, we examined the effect of various reaction temperatures on the mutant reverse transcriptases from 50° C. to 75° C. A special program entailed gradient temperatures from 56° C. to 66.5° C. for 10 minutes for reverse transcription step.

RT-PAP Assay IV is a larger and more difficult target for reverse transcription, and it reflects the optimal temperature more clearly than RT-PAP Assay III.

With RT-PAP Assay IV, each mutant reverse transcriptase was used to measure Cₜ values at different temperatures (Right part of Table 8). Because Cₜ values are proportional to the reverse transcriptase activities or efficiencies, the Cₜ values can represent them. For comparison of the Cₜ values at different temperatures, a ΔCₜ is set which equals to a Cₜ value subtracts that at 60.1° C. (Right part of Table 8). The results showed that the reverse transcriptases catalyzed both RNA-dependent DNA pyrophosphorolysis and reverse transcription in the RT-PAP assays substantially from 56° C. to 66.5° C. (Left part of Table 8). At 60.1-62.7° C., the Cₜ values were earliest and the ΔCₜ values were the smallest, indicating the reverse transcriptase activities or efficiencies to be highest at 60.1-62.7° C.

In addition, other tests also showed that the reverse transcriptases still had observable activities at 55° C. and at 70° C.

The reverse transcriptases showed similar properties of the optimal temperatures even with as low as 85% identity among their amino acid sequences (Tables 1 and 2) and as low as 90% identity among their DNA polymerase A domains (Tables 3 and 4).

### Specific Activity

In order to compare relative activities among the mutant reverse transcriptases, the specific activity is defined as the minimum amount (ng) of a reverse transcriptase that has an almost 100% efficiency in reverse transcription step.

RT-PAP Assay IV was chosen because it is a larger and more difficult target and measures the level of reverse transcriptase activity more clearly than RT-PAP Assay III.

We tested and compared the specific activities of the mutant reverse transcriptases. The amount of each reverse transcriptase was 2-fold serially diluted from up to 200 ng to 1 ng in 20 μl of reaction. The Cₜ value was determined to estimate its minimum amount of the reverse transcriptase with an almost 100% efficiency of reverse transcription (Right part of Table 9). For comparison of the relative activities among the reverse transcriptases, the ratio of the minimum amount of a reverse transcriptase to that of Cpe-665Y was counted (Right part of Table 9). The mutant reverse transcriptases showed various specific activities with Cpe-665Y DNA polymerase having the highest specific activity among them.

In melting curve analysis, only one melting peak showed the Tₘ value to be 86° C. of the amplified product in RT-PAP Assay IV. Non-template-control did not show any measurable Ct, showing specificity of the amplification.

In addition, when RT-PAP Assay III was used, the specific activities were higher with less amounts of the mutant reverse transcriptases required than those in RT-PAP Assay IV.

The reverse transcriptases showed various specific activities within a limited range, depending not only on the particular reverse transcriptases but also on the particular assays.

### RT-PAP Assays for Detection of COVID-19 Viral RNA

RT-PAP assays were developed to detect COVID-19 viral RNA for clinical diagnostics.

Specifically, we used RT-PAP Assay V to amplify from an RNA target in the N region of the COVID-19 viral genome in a first tube with two 3′ blocked primers of COVID-19(28679)28D and COVID-19(28754)28U (SEQ ID NOS: 25 and 26). The RT-PAP Assay V includes two steps of RT and PAP. 1) Using the COVID-19 viral RNA template, a reverse transcriptase (Cpe-665Y) removes the 3′ blocker from the 3′ blocked primer [COVID-19(28754)28U, SEQ ID NO: 26], and then extends the unblocked primer to generate a DNA product. 2) Using the DNA product as template, a DNA-dependent DNA polymerase (TaqFS) removes the 3′ blockers from the 3′ blocked primers [COVID-19(28679)28D and COVID-19(28754)28U, SEQ ID NOS: 25 and 26]. The amplified product was measured by real-time fluorescence signals emitted by SYBR Green (EXAMPLE 3).

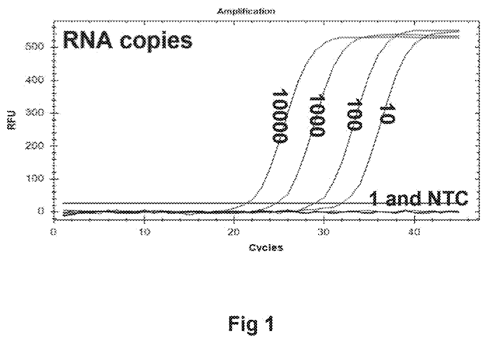

The sensitivity of RT-PAP Assay V was validated. In an experiment, the amount of COVOD-19 RNA template was 10-fold serially diluted with detection limit estimated to be a single digit of copies of RNA template (FIG. 1).

In addition, we used RT-PAP Assay VI to amplify from a control RNA of the human ACTB gene in a second tube with two 3′ blocked primers of ACTB(434)28D and ACTB(512)27U (SEQ ID NOS: 27 and 28).

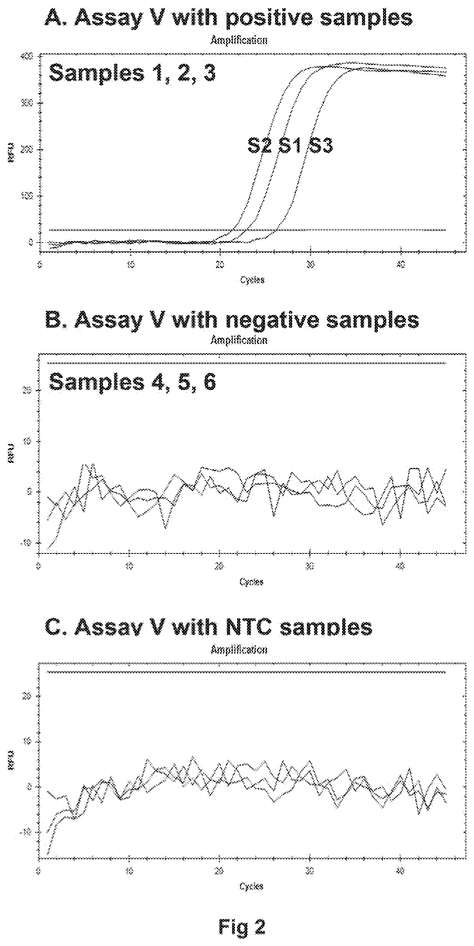

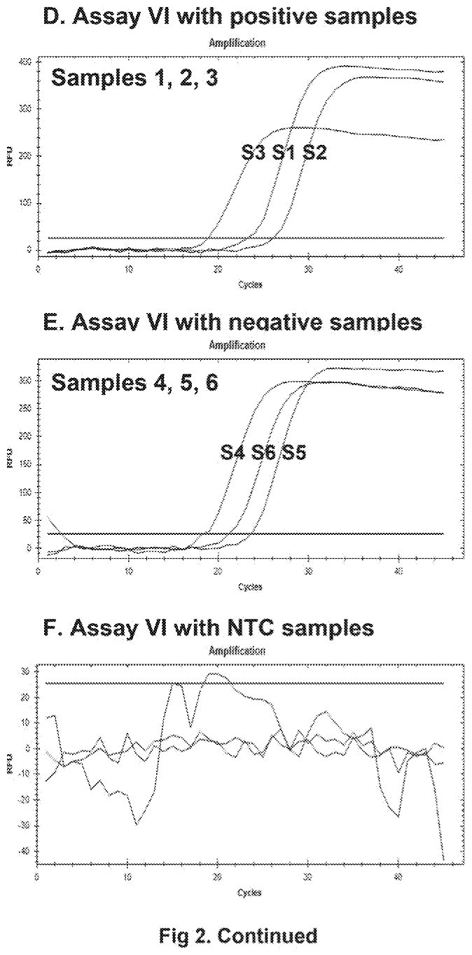

The application to clinical samples was demonstrated for RT-PAP Assay V to amplify from a potential COVOD-19 viral RNA template and for RT-PAP Assay VI to amplify from a control RNA template of the human ACTB gene. COVOD-19 positive, negative and non-template-control samples were tested and showed true positive and true negative results. In addition, no false positives and no false negatives were observed (FIG. 2).

### Example 6

Other compositions and their concentrations, such as reaction buffer compositions and their concentrations, Magnesium concentrations and dNTP concentrations, were optimized together with the cycling programs, as described in EXAMPLE 3.

### REFERENCE

Arion, D., Kaushik, N., McCormick, S., Borkow, G., and Parniak, M. A. (1998). Phenotypic mechanism of HIV-1 resistance to 3′-azido-3′-deoxythymidine (AZT): increased polymerization processivity and enhanced sensitivity to pyrophosphate of the mutant viral reverse transcriptase. Biochemistry 37, 15908-15917.
Ausubel, F. M. (1994). Current protocols in molecular biology, John Wiley & Sons, New York
Deutscher, M. P., and Kornberg, A. (1969). Enzymatic synthesis of deoxyribonucleic acid. 28. The pyrophosphate exchange and pyrophosphorolysis reactions of deoxyribonucleic acid polymerase. The Journal of biological chemistry 244, 3019-3028.
Gardner, A. F., Joyce, C. M., and Jack, W. E. (2004). Comparative kinetics of nucleotide analog incorporation by vent DNA polymerase. The Journal of biological chemistry 279, 11834-11842.
Jones, M. D., and Foulkes, N. S. (1989). Reverse transcription of mRNA by Thermus aquaticus DNA polymerase. Nucleic acids research 17, 8387-8388.
Myers, T. W., and Gelfand, D. H. (1991). Reverse transcription and DNA amplification by a Thermus thermophilus DNA polymerase. Biochemistry 30, 7661-7666.
Liu, Q., and Sommer, S. S. (2000). Pyrophosphorolysis-activated polymerization (PAP): application to allele-specific amplification. BioTechniques 29, 1072-1080.
Liu, Q., and Sommer, S. S. (2002). Pyrophosphorolysis-activatable oligonucleotides may facilitate detection of rare alleles, mutation scanning and analysis of chromatin structures. Nucleic acids research 30, 598-604.
Liu, Q., and Sommer, S. S. (2004a). Pyrophosphorolysis by Type II DNA polymerases: implications for pyrophosphorolysis-activated polymerization. Analytical biochemistry 324, 22-28.
Liu, Q., and Sommer, S. S. (2004b). Detection of extremely rare alleles by bidirectional pyrophosphorolysis-activated polymerization allele-specific amplification (Bi-PAP-A): measurement of mutation load in mammalian tissues. BioTechniques 36, 156-166.
Sambrook, J., Fritsch, E. F., and Maniatis, T. (1989). Molecular cloning, a laboratory manual, second edition, Cold Spring Harbor Laboratory Press, Cold Spring Harbor, New York
Sambrook, J., and Russell, D. W. (2001). Molecular Cloning: A Laboratory Manual, Third Edition, Cold Spring Harbor Laboratory Press, Cold Spring Harbor, New York.
Smith, E. S. E., Elfstrom, C. M., Gelfand, D. H., Higuchi, R. G., Myers, T. W., Schonbrunner, N. J., and Wang, A. M. (2007). High temperature reverse transcription using mutant DNA polymerases. U.S. Pat. No. 7,179,590.
Verma, I. M. (1977). The reverse transcriptase. Biochimica et biophysica acta 473, 1-38.
Urban, S., Fischer, K. P., and Tyrrell, D. L. (2001). Efficient pyrophosphorolysis by a hepatitis B virus polymerase may be a primer-unblocking mechanism. Proceedings of the National Academy of Sciences of the United States of America 98, 4984-4989.

1. A thermophilic purified DNA-dependent DNA polymerase from Carboxydothermus pertinax that catalyzes reverse transcription or RNA-dependent DNA polymerization on an RNA template, wherein the polymerase is also a reverse transcriptase or an RNA-dependent DNA polymerase, comprising an amino acid sequence with more than 85% sequence identity to SEQ ID NO: 1, and wherein the reverse transcriptase activity is heat-inactivated at a temperature ≥83° C.

2. The thermophilic purified DNA-dependent DNA polymerase from Carboxydothermus pertinax that catalyzes reverse transcription or RNA-dependent DNA polymerization on an RNA template of claim 1, wherein the reverse transcriptase activity is heat-inactivated ≥90% at a temperature ≥83° C. for 2 min.

3. The thermophilic purified DNA-dependent DNA polymerase from Carboxydothermus pertinax that catalyzes reverse transcription or RNA-dependent DNA polymerization on an RNA template of claim 1, wherein the polymerase is selected from a group of polymerases consisting of SEQ ID NOs: 1-8.

4. The thermophilic purified DNA-dependent DNA polymerase from Carboxydothermus pertinax that catalyzes reverse transcription or RNA-dependent DNA polymerization on an RNA template of claim 1, wherein the polymerase comprises a DNA polymerase A domain which amino acid sequence is more than 90% sequence identity to SEQ ID NO: 1.

5. The thermophilic purified DNA-dependent DNA polymerase from Carboxydothermus pertinax that catalyzes reverse transcription or RNA-dependent DNA polymerization on an RNA template of claim 1, wherein the polymerase comprises a DNA polymerase A domain which is selected from a group of DNA polymerase A domains consisting of SEQ ID NOs: 1-8.

6. The thermophilic purified DNA-dependent DNA polymerase from Carboxydothermus pertinax that catalyzes reverse transcription or RNA-dependent DNA polymerization on an RNA template of claim 1, wherein the reverse transcriptase activity is heat-inactivated at a temperature from 83° C. to 95° C.

7. The thermophilic purified DNA-dependent DNA polymerase from Carboxydothermus pertinax that catalyzes reverse transcription or RNA-dependent DNA polymerization on an RNA template of claim 1, wherein the reverse transcriptase catalyzes reverse transcription at a temperature from 55° C. to 70° C.

8. The thermophilic purified DNA-dependent DNA polymerase from Carboxydothermus pertinax that catalyzes reverse transcription or RNA-dependent DNA polymerization on an RNA template of claim 1, wherein the reverse transcriptase catalyzes reverse transcription at an optimal temperature from 60° C. to 62° C.

9. The thermophilic purified DNA-dependent DNA polymerase from Carboxydothermus pertinax that catalyzes reverse transcription or RNA-dependent DNA polymerization on an RNA template of claim 1, wherein (a) the reverse transcriptase catalyzes reverse transcription on the RNA template to generate a reverse-transcribed DNA, and (b) a second DNA polymerase catalyzes DNA-dependent DNA polymerization using the reverse-transcribed DNA as template.

10. A thermophilic purified DNA-dependent DNA polymerase that catalyzes RNA-dependent DNA pyrophosphorolysis on an RNA template, wherein the polymerase is also a reverse transcriptase or an RNA-dependent DNA polymerase, comprising an amino acid sequence with more than 85% sequence identity to DNA-dependent DNA polymerase of SEQ ID NO: 1.

11. The thermophilic purified DNA-dependent DNA polymerase that catalyzes RNA-dependent DNA pyrophosphorolysis on an RNA template of claim 10, wherein the reverse transcriptase contains a phenylalanine to tyrosine substitution corresponding to amino acid position 665 of SEQ ID NO: 1.

12. The thermophilic purified DNA-dependent DNA polymerase that catalyzes RNA-dependent DNA pyrophosphorolysis on an RNA template of claim 10, wherein the reverse transcriptase is selected from SEQ ID NO: 1, SEQ ID NO: 2, SEQ ID NO: 3, SEQ ID NO: 4, SEQ ID NO: 5, SEQ ID NO: 6, SEQ ID NO: 7, or SEQ ID NO: 8.

13. The thermophilic purified DNA-dependent DNA polymerase that catalyzes RNA-dependent DNA pyrophosphorolysis on an RNA template of claim 10, wherein the polymerase comprises a DNA polymerase A domain which amino acid sequence has more than 90% sequence identity to DNA-dependent DNA polymerase of SEQ ID NO: 1.

14. The thermophilic purified DNA-dependent DNA polymerase that catalyzes RNA-dependent DNA pyrophosphorolysis on an RNA template of claim 10, wherein the reverse transcriptase comprises a DNA polymerase A domain which is selected from SEQ ID NO: 1, SEQ ID NO: 2, SEQ ID NO: 3, SEQ ID NO: 4, SEQ ID NO: 5, SEQ ID NO: 6, SEQ ID NO: 7 or SEQ ID NO: 8.

15. The thermophilic purified DNA-dependent DNA polymerase that catalyzes RNA-dependent DNA pyrophosphorolysis on an RNA template of claim 10, wherein when a 3′ blocked primer that has a non-extendable nucleotide at the 3′ blocker anneals to the RNA template, the reverse transcriptase catalyzes RNA-dependent DNA pyrophosphorolysis to remove the 3′ blocker from the 3′ blocked primer.

16. The thermophilic purified DNA-dependent DNA polymerase that catalyzes RNA-dependent DNA pyrophosphorolysis on an RNA template of claim 15, wherein the reverse transcriptase catalyzes RNA-dependent DNA polymerization on the RNA template to extend the shortened 3′ blocked primer.

17. The thermophilic purified DNA-dependent DNA polymerase that catalyzes RNA-dependent DNA pyrophosphorolysis on an RNA template of claim 10, wherein the reverse transcriptase activity is heat-inactivated at a temperature ≥83° C.

18. The thermophilic purified DNA-dependent DNA polymerase that catalyzes RNA-dependent DNA pyrophosphorolysis on an RNA template of claim 10, wherein the reverse transcriptase activity is heat-inactivated at a temperature from 83° C. to 95° C.

19. The thermophilic purified DNA-dependent DNA polymerase that catalyzes RNA-dependent DNA pyrophosphorolysis on an RNA template of claim 10, wherein the reverse transcriptase catalyzes RNA-dependent DNA pyrophosphorolysis at a temperature from 55° C. to 70° C.

20. The thermophilic purified DNA-dependent DNA polymerase that catalyzes RNA-dependent DNA pyrophosphorolysis on an RNA template of claim 10, wherein the reverse transcriptase catalyzes RNA-dependent DNA pyrophosphorolysis at an optimal temperature from 60° C. to 62° C.
